(19) United States
(12) Patent Application Publication
(10) Pub. No.: US 2012/0331575 A1
(43) Pub. Date: Dec. 27, 2012

(54) TRANSGENIC NON-HUMAN ANIMALS

(75) Inventors: Peter Hobman, Melbourne (AU); Matthew McDonagh, Williamstown (AU); Benjamin Cocks, Viewbank (AU); Angus Tester, Moonee Ponds (AU)

(73) Assignees: Agriculture Victoria Services PTY LTD, Attwood (AU); Murray Goulburn Co-Operative Co. Limited, Brunswick (AU)

(21) Appl. No.: 13/510,171

(22) PCT Filed: Nov. 18, 2010
(86) PCT No.: PCT/AU2010/001542

§ 371 Date: Sep. 5, 2012

(30) Foreign Application Priority Data
Nov. 18, 2009 (AU) 2009905629

(51) Int. Cl. A01K 67/027; A61K 38/19; A61P 21/00; A61P 19/10; A61P 19/08; A23L 1/32; A61P 29/00; A61P 3/06; A61D 19/04; A23C 9/00; A23L 1/31; C12N 15/85; A61P 25/00

(52) U.S. Cl. 800/14; 435/320.1; 514/1.1; 600/34; 426/2; 426/531; 426/580; 426/641; 426/614

(57) ABSTRACT

The invention provides a non-human transgenic animal comprising a transgene encoding angiogenin and food products comprising or obtained from the non-human transgenic animal and uses thereof.

### FIELD

The present invention is in the field of transgenic non-human animals.

### BACKGROUND

Angiogenin, encoded by the ANG gene, is a member of the ribonuclease (RNase) superfamily. Angiogenin (also known as RNase5) is a 14 kDa, non-glycosylated secreted ribonuclease polypeptide. Angiogenin is known to regulate the formation of new blood vessels through a process called angiogenesis and is known to regulate neuron survival, with functional mutations in the protein being a cause of the neuromuscular disorder amyotrophic lateral sclerosis (ALS).

During angiogenisis, the angiogenin protein binds to receptors on the surface of endothelial cells and smooth muscle cells and undergoes nuclear translocation where it stimulates the production of ribosomal RNA (rRNA) which is required for the growth and division of cells for capillary formation. Angiogenesis associated with exercise causes capillary growth that allows for greater nutrient and oxygen delivery to muscle tissue.

In our co-pending application PCT/AU2009/000603 we demonstrated that angiogenin increases muscle cell growth and differentiation in vitro, and significantly alleviates the potent inhibitory effects of myostatin on myoblasts. Angiogenin is enriched in colostrum and milk, secretions which evolved to promote health, growth and development of suckling mammals. When added to the feed of mice, angiogenin purified from bovine milk increased exercising muscle growth by 50% over a 4 week period. We demonstrated that angiogenin is bioavailable when administered orally in our co-pending application PCT/AU2009/000602.

Angiogenin has also been shown to possess a number of other activities. These include the ability to remove skin defects such as pigmented spots, modulation of immune responses, protection of polymorphonuclear leukocytes from spontaneous degradation, and microbicidal activity against systemic bacterial and fungal pathogens. Angiogenin also appears to be required for effective activity of growth factors such as VEGF, EGF and FGF. In addition, functional mutations in the angiogenin protein cause the neuromuscular disorder amyotrophic lateral sclerosis (ALS).

Angiogenin may have numerous applications, including applications in medicine, dietary foodstuff supplements and cosmetics. However, the use of angiogenin in such applications requires an efficient process for the preparation of the protein on a commercial scale from an appropriate source.

Angiogenin is readily available in bovine milk, however its use as a source of angiogenin is not favoured as angiogenin is only present in bovine milk at a low level. Also, certain proteins present in milk, such as caseins, and milk whey proteins such as immunoglobulin, lactoferrin and lactoperoxidase, mask angiogenin, hindering its purification.

Reference to any prior art in the specification is not, and should not be taken as, an acknowledgment or any form of suggestion that this prior art forms part of the common general knowledge in Australia or any other jurisdiction or that this prior art could reasonably be expected to be ascertained, understood and regarded as relevant by a person skilled in the art.

It is an aim of an embodiment of the present invention to overcome, or at least alleviate, one or more of the difficulties or deficiencies associated with the prior art.

### SUMMARY

The present invention in a first aspect provides a non-human transgenic animal that includes a transgene encoding angiogenin.

The transgenic animal may also include a transgene encoding follistatin or the transgenic animal may include a transgene encoding angiogenin and follistatin.

In some embodiments, the transgenic animal is a mammal. In some embodiments, the transgenic mammal is an ungulate. In other embodiments, the transgenic animal is a poultry animal. In many embodiments, the transgene is chromosomally integrated. In many embodiments, the transgene includes a coding sequence for angiogenin, operably linked to an animal tissue specific promoter. In some embodiments, the animal tissue specific promoter is a mammary specific promoter. In other embodiments, the animal tissue specific promoter is a muscle specific promoter or a liver specific promoter. In many embodiments, a tissue of a subject transgenic animal (e.g., milk, meat, or egg) has a level of angiogenin that is at least 5% higher than the level of angiogenin in a control non-transgenic animal of the same species.

The invention further provides an expression cassette comprising a coding sequence for angiogenin operably linked to a heterologous mammalian tissue-specific promoter. In some embodiments, the heterologous tissue specific promoter is a mammary specific promoter. In other embodiments, the heterologous tissue specific promoter is a muscle promoter. In many embodiments, the expression cassette is present in a vector.

Other than use to produce transgenic animals the expression cassette could be used in gene therapy or as a DNA vaccine. Particularly the expression cassette could be included in an adenovirus vector. Such approaches may provide a muscle or systemic effect of angiogenin.

The invention further provides a method for producing the non-human transgenic animal of the first aspect. The method generally involves introducing an angiogenin transgene of the first aspect into a single-celled embryo, forming a genetically modified embryo; and transferring the genetically modified embryo into a recipient female of the same species as the embryo, wherein the genetically modified embryo develops into a transgenic animal in the female. In some embodiments, the methods involve introducing a transgene into a cell, generating a genetically modified cell with a genetically modified nucleus; transferring the nucleus of the genetically modified cell into an oocyte or a single-celled embryo, generating a genetically modified oocyte or a genetically modified single-celled embryo; and transferring the genetically modified oocyte or genetically-modified single-celled embryo into a recipient female of the same species, where the genetically modified oocyte or genetically-modified single-celled embryo develops into a transgenic animal in the recipient female. In the present methods, the transgenic animal is chosen from a mouse, a rat, a rabbit, a pig, a sheep, a goat, poultry animal, a cow and a horse. In some embodiments, the transgenic animal is a mammal, and the transgene is expressed in mammary gland cells of the mammal. In other embodiments, the transgenic animal is a mammal, and the transgene is expressed in muscle cells of the mammal. In other embodiments, the transgenic animal is a poultry animal, and the transgene is expressed in intestinal epithelium cells and/or intestinal glandular tissue of the poultry animal.

Other embodiments which involve selectively enhancing expression of the endogenous angiogenin gene and optionally the endogenous follistatin gene are contemplated.

The present invention further provides a method of producing a food product, feedstock, food supplement or veterinary product. In some embodiments, the method generally involves harvesting the food product, from a subject non-human transgenic animal. In other embodiments, the method generally involves processing a food product harvested from a subject non-human transgenic animal.

The present invention further provides a food product, feedstock, food supplement or veterinary product harvested from a subject non-human transgenic animal. In some embodiments, the food product is processed. In some embodiments, the food product is milk. In other embodiments, the food product is meat. In other embodiments, the food product is an egg. The food product may be intended for human consumption or may be used as animal feed. Consumption of such foods by livestock animals increases the rate of growth of such animals, and increases the feed efficiency.

The present invention further provides use of the subject non-human transgenic animal as a food product or food source.

The present invention further provides use of the subject non-human transgenic animal as a source of angiogenin and optionally follistatin, particularly when the angiogenin is produced in milk from the transgenic animal. The angiogenin may be purified using techniques known in the art, for example cation exchange chromatography, immunoaffinity chromatography, ultrafiltration or size exclusion, for example as outlined in our co-pending applications PCT/AU2007/001719 and PCT/AU2009/000604).

Said angiogenin and optionally follistatin may be used for treatment of disease in animals, particularly humans. Diseases to be treated include those described in PCT/AU2009/000603, such as muscle disorders, including muscle wasting disorders, muscular dystrophy, muscular atrophy, sarcopenia, cachexia, improving muscle form by improving muscle strength, mass or exercise tolerance, decreasing fat, improving muscle to fat ratio, treating diseases caused by or involving suboptimal muscle to fat ratio which effect is enhanced by follistatin, treating bone disorders including osteoporosis, improving bone density, treating neurological disorders or diseases affecting the nervous system, particularly motor neurone diseases such as ALS, spinal muscular atrophys, inflammation myopathies including dermatomyositis, polymyositis and inclusion body myositis, diseases of the neuromuscular junction, such as Myasthenia Gravis (MG), Lambert-Eaton Syndrome (LES), and Congenital Myasthenic Syndrome (CMS), myopathies due to endocrine abnormalities, such as Hyperthyroid Myopathy (HYPTM) and Hypothyroid Myopathy (HYPOTM), diseases of peripheral nerve such as Charcot-Marie-Tooth Disease (CMT), Dejerine-Sottas Disease (DS), and Friedreich's Ataxia (FA), other myopathies including Myotonia Congenita (MC), Paramyotonia Congenita (PC), Central Core Disease (CCD), Nemaline Myopathy (NM), Myotubular Myopathy (MTM or MM), and Periodic Paralysis (PP), wound healing, metabolic diseases of muscle, including Phosphorylase Deficiency (MPD or PYGM), Acid Maltase Deficiency (AMD), Phosphofructokinase Deficiency (PFKM), Debrancher Enzyme Deficiency (DBD), Mitochondrial Myopathy (MITO), Carnitine Deficiency (CD), Carnitine Palmityl Transferase Deficiency (CPT), Phosphoglycerate Kinase Deficiency (PGK), Phosphoglycerate Mutase Deficiency (PGAM or PGAMM), Lactate Dehydrogenase Deficiency (LDHA), and Myoadenylate Deaminase Deficiency (MAD), diseases connected to impaired lipid metabolism such as dyslipidemia and related lipid abnormalities such as hyperlipidemia, hypercholesteremia, hypertriglyceridemia and mixed dyslipidemia, spine injuries or diseases, diseases involving glucose homeostasis, for providing neuroprotection, nervous system functional support and managing metabolic diseases and diseases connected to impaired glucose metabolism and impaired insulin action including diabetes mellitus, especially diabetes mellitus type 1 and 2, non-autoimmune non-insulin dependent diabetes mellitus, syndrome X or metabolic syndrome.

The angiogenin from a transgenic non-human animal may also be used for microbial inhibition, enhancing gut epithelial function, wound healing, and bacterial flora symbiosis and potentially ingesting foods containing angiogenin may have beneficial effects on gut health and gut based disease prevention and immune enhancement in humans and livestock animals. Given the in vivo effects of oral angiogenin described in PCT/AU2009/000602 on mouse muscle and the role in regulating protein synthesis in muscle, administration of angiogenin would be expected to enhance muscle production in livestock animals. Given the conserved function of angiogenin in vertebrates in regulation of angiogenesis and activity when used across wide species boundaries, angiogenin is expected to have a role in the development of broiler chicken gut, immunity, muscle and growth and maintain health of chicken layers to enhance egg laying productivity.

For humans and companion animals, animal health and muscle composition can be improved and the above mentioned diseases can be treated or prevented by ingestion of angiogenin produced from transgenic non-human animals.

The transgenic non-human animals of the invention provide a ready source of angiogenin for use in pharmaceuticals, nutraceuticals and functional foods for treating or preventing the above mentioned diseases.

The present invention further provides for use of the subject non-human transgenic animal as a model for studying diseases involving angiogenin dysfunction and for identifying modulators of angiogenin and potential therapeutic candidates.

The invention further provides a method of increasing the muscle mass of a non-human animal by making that animal transgenic for angiogenin and optionally follistatin.

Particularly the invention provides a transgenic non-human animal having a phenotype characterized by muscle hyperplasia, said phenotype being conferred by a transgene contained in the somatic and germ cells, the transgene encoding angiogenin and optionally follistatin.

The invention further provides embryonic stem cells or somatic cells from a non-human animal comprising a transgene encoding angiogenin and optionally follistatin for nuclear transfer to produce a non-human animal transgenic for angiogenin and optionally follistatin.

The invention further provides eggs, embryos and sperm from the transgenic non-human animals of the invention.

In a further aspect, the present invention provides a food, beverage, food supplement, nutraceutical or pharmaceutical including angiogenin produced from the transgenic animal of the first aspect.

### DETAILED DESCRIPTION

The present invention provides transgenic, non-human animals that include a transgene that encodes angiogenin and optionally follistatin and methods for producing such animals. The subject transgenic animals fall into at least two categories, depending on where the angiogenin is to be expressed:
1. Those producing transgenic milk or eggs as a source of angiogenin; and
2. Those with improved carcass composition and muscle hyperplasia, to provide food such as meat.

Subject transgenic animals have increased levels of angiogenin. In our co-pending applications PCT/AU2009/000602 and PCT/AU2009/000603 we demonstrate that angiogenin has an effect on muscle metabolism by oral administration and propose that angiogenin can be useful in treating muscle disorders, including muscle wasting disorders, muscular dystrophy, muscular atrophy, sarcopenia, cachexia, improving muscle form by improving muscle strength, mass or exercise tolerance, decreasing fat, improving muscle to fat ratio, treating diseases caused by or involving suboptimal muscle to fat ratio which effect is enhanced by follistatin, treating bone disorders including osteoporosis, improving bone density, treating neurological disorders or diseases affecting the nervous system, particularly motor neurone diseases such as ALS, spinal muscular atrophys, inflammation myopathies including dermatomyositis, polymyositis and inclusion body myositis, diseases of the neuromuscular junction, such as Myasthenia Gravis (MG), Lambert-Eaton Syndrome (LES), and Congenital Myasthenic Syndrome (CMS), myopathies due to endocrine abnormalities, such as Hyperthyroid Myopathy (HYPTM) and Hypothyroid Myopathy (HYPOTM), diseases of peripheral nerve such as Charcot-Marie-Tooth Disease (CMT), Dejerine-Sottas Disease (DS), and Friedreich's Ataxia (FA), other myopathies including Myotonia Congenita (MC), Paramyotonia Congenita (PC), Central Core Disease (CCD), Nemaline Myopathy (NM), Myotubular Myopathy (MTM or MM), and Periodic Paralysis (PP), wound healing, metabolic diseases of muscle, including Phosphorylase Deficiency (MPD or PYGM), Acid Maltase Deficiency (AMD), Phosphofructokinase Deficiency (PFKM), Debrancher Enzyme Deficiency (DBD), Mitochondrial Myopathy (MITO), Carnitine Deficiency (CD), Carnitine Palmityl Transferase Deficiency (CPT), Phosphoglycerate Kinase Deficiency (PGK), Phosphoglycerate Mutase Deficiency (PGAM or PGAMM), Lactate Dehydrogenase Deficiency (LDHA), and Myoadenylate Deaminase Deficiency (MAD), diseases connected to impaired lipid metabolism such as dyslipidemia and related lipid abnormalities such as hyperlipidemia, hypercholesteremia, hypertriglyceridemia and mixed dyslipidemia, spine injuries or diseases, diseases involving glucose homeostasis, for providing neuroprotection, nervous system functional support and managing metabolic diseases and diseases connected to impaired glucose metabolism and impaired insulin action including diabetes mellitus, especially diabetes mellitus type 1 and 2, non-autoimmune non-insulin dependent diabetes mellitus, syndrome X or metabolic syndrome.

The angiogenin from a transgenic non-human animal may also be used for microbial inhibition, enhancing gut epithelial function, wound healing, and bacterial flora symbiosis and potentially ingesting foods containing angiogenin may have beneficial effects on gut health and gut based disease prevention and immune enhancement in humans and livestock animals.

In the context of the invention described herein the main effect of angiogenin sought in relation to livestock animals is the improvement of muscle mass and muscle to fat ratio to provide improved carcass composition, whereas if the angiogenin is targeted to the mammary gland, or to the egg production gland, the aim of the invention is to provide a ready source of angiogenin in milk or eggs, optionally at increased yield. The milk or eggs can then be used as a functional food or nutraceutical, providing angiogenin to persons in need thereof. Alternatively angiogenin can be purified from the milk or eggs, for example using the methods described in PCT/AU2007/001719 and PCT/AU2009/000604 and used in pharmaceutical, veterinary, nutraceutical and functional food formulations.

The following list defines terms, phrases and abbreviations used throughout the specification. Although the terms, phrases and abbreviations are listed in the singular tense, this list is intended to encompass all grammatical forms.

As used herein, except where the context requires otherwise, the term “comprise” and variations of the term, such as “comprising”, “comprises” and “comprised”, are not intended to exclude further additives, components, integers or steps.

As used herein, the term “transgenic” refers to a cell and/or animal having a genome into which genetic material from a different organism has been artificially introduced.

The term “transgene” is used herein to describe genetic material which has been or is about to be artificially inserted into the genome of a non-human animal, and particularly into a cell of a living non-human mammal, or into a cell of a living bird.

As used herein, the term “expression” includes transcription and translation.

As used herein, the term “heterologous” or “foreign” refers to nucleic acid and/or amino acid sequences not naturally occurring in the cell/organism of interest. Heterologous sequences may also be found in a location or locations in the genome that differs from that in which it occurs in nature.

As used herein, the term “endogenous” refers to nucleic acid and/or amino acid sequences naturally occurring in the cell/organism of interest.

As used herein, the term “recombinant” refers to genetic material, cells and/or organisms that have been genetically modified; for example, by addition of heterologous genetic material or modification of the endogenous genetic material.

As used herein, the term “isolated” or “purified” refers to nucleic acid and/or amino sequences that have been removed from at least one component with which it is naturally associated. For example, an isolated protein is substantially free of cellular material or culture medium when produced by molecular biological techniques.

As used herein, the term “vector” refers to a polynucleotide construct designed for transduction and/or transfection of one or more cell types.

As used herein, the phrase “operably linked” when referring to a transcriptional regulatory element and a coding sequence is intended to mean that the regulatory sequence is associated with the coding sequence in such a manner as to facilitate transcription of the coding sequence.

As used herein, the term “homologous recombination” refers to the exchange of DNA fragments between two DNA molecules or chromatids at the site of homologous nucleotide sequences.

As used herein, the term “gene targeting” refers to a type of homologous recombination that occurs when a fragment of genomic DNA is introduced into a mammalian cell and that fragment locates and recombines with endogenous homologous sequences.

As used herein, the term “double-muscling” describes an increase in skeletal muscle mass. Double-muscling can result from muscular hyperplasia and/or hypertrophy.

As used herein, the term “hyperplasia” refers to an abnormal increase in the number of cells in an organ and/or tissue resulting in enlargement of the organ and/or tissue.

As used herein, the term “hypertrophy” refers to the enlargement of an organ and/or tissue resulting from an increase in the size of the individual cells of the organ and/or tissue.

As used herein, the term “genotype” refers to the entire genetic constitution of an organism; i.e. genes of an organism, both dominant and recessive.

As used herein, the term “phenotype” refers to the observable characteristics of an individual resulting from the interaction of the individual's genotype with the environment.

As used herein, the term “allele” refers to an alternative form of a gene and/or any one of several mutational forms.

As used herein, the term “promoter” refers to a sequence at the 5′ end of a gene which binds DNA polymerase and/or transcription factors to regulate expression of the gene. Promoters can be tissue-specific.

The term “transformation” refers to a permanent or transient genetic change induced in a cell following the incorporation of new DNA (i.e. DNA exogenous to the cell). Where the cell is a mammalian cell or an avian cell, a permanent genetic change is generally achieved by introduction of the DNA into the genome of the cell.

The term “ES cell” as used herein refers to pluripotent embryonic stem cells and to such pluripotent cells in the very early stages of embryonic development, including but not limited to cells in the blastocyst stage of development.

The term “construct” refers to a recombinant nucleic acid, generally recombinant DNA, that has been generated for the purpose of the expression of a specific nucleotide sequence(s), or is to be used in the construction of other recombinant nucleotide sequences.

The term “cDNA” refers to all nucleic acids that share the arrangement of sequence elements found in native mature mRNA species, where sequence elements are exons and 3′ and 5′ non-coding regions. Normally mRNA species have contiguous exons, with the intervening introns removed by nuclear RNA splicing, to create a continuous open reading frame encoding the protein.

The term “genomic sequence” refers to a sequence having non-contiguous open reading frames, where introns interrupt the protein coding regions. It may further include the 3′ and 5′ untranslated regions found in the mature mRNA. It may further include specific transcriptional and translational regulatory sequences, such as promoters, enhancers, etc., including about 1 kb, but possibly more, of flanking genomic DNA at either the 5′ or 3′ end of the transcribed region. The genomic DNA may be isolated as a fragment of 100 kbp or smaller; and substantially free of flanking chromosomal sequence.

The invention extends to angiogenin extracted from milk, eggs or meat of transgenic non-human animals.

The invention in one aspect relates to the treatment of disorders. The terms “treating” and “treatment” as used herein refer to reduction in severity and/or frequency of symptoms, elimination of symptoms and/or underlying cause, prevention of the occurrence of symptoms (prophylaxis) and/or their underlying cause, and improvement or remediation of damage. Thus, for example, the present method of “treating” a disorder encompasses both prevention of the disorder in a predisposed individual and treatment of the disorder in a clinically symptomatic individual.

“Treating” as used herein covers any treatment of, or prevention of a condition in a vertebrate, a mammal, particularly a human, and includes: inhibiting the condition, i.e., arresting its development; or relieving or ameliorating the effects of the condition, i.e., cause regression of the effects of the condition.

“Prophylaxis” or “prophylactic” or “preventative” therapy as used herein includes preventing the condition from occurring or ameliorating the subsequent progression of the condition in a subject that may be predisposed to the condition, but has not yet been diagnosed as having it.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a”, “and”, and “the” include plural referents unless the context clearly dictates otherwise. Thus, for example, reference to “a transgenic non-human animal” includes a plurality of such animals and reference to “the transgene” includes reference to one or more transgenes and equivalents thereof known to those skilled in the art, and so forth.

In further describing the subject invention, the subject transgenic animals and methods for their production are described first in greater detail, followed by a review of representative applications to which the subject animals find use, e.g., in food production, etc.

Transgenic Non-Human Animals and Methods for their Production

The present invention provides transgenic non-human animals that include an angiogenin transgene. An angiogenin transgene includes a nucleotide sequence that encodes angiogenin. In many embodiments, the angiogenin coding sequence is operably linked to a tissue-specific promoter such that the encoded angiogenin is produced in a tissue-specific manner, e.g., in the mammary gland or in muscle cells. As follistatin is considered to enhance angiogenin activity the transgenic animal or the angiogenin transgene may also include a follistatin transgene.

The description provided herein as it relates to angiogenin transgenes and transgenic animals is meant to be exemplary only, and is not meant to be limited to particular angiogenin transgenes and transgenic animals. Any angiogenin transgene can be used to generate a subject transgenic animal, provided that the subject transgenic animal exhibits increased muscle mass if the transgene is targeted to the muscle or increased yield of angiogenin in milk if the transgene is targeted to the mammary gland when compared to a non-transgenic littermate.

A subject transgenic animal that expresses an angiogenin transgene in muscle has increased muscle size compared to a non-transgenic littermate. For example, the muscle mass in a subject transgenic animal, is at least about 2.5%, at least about 5%, at least about 10%, at least about 15%, at least about 20%, at least about 25%, at least about 30%, at least about 35%, at least about 40%, at least about 45%, or at least about 50% higher than the level in a control animal, e.g., a non-transgenic animal of the same species, such as a non-transgenic littermate.

A subject transgenic animal that expresses an angiogenin transgene in mammary glands has increased angiogenin in its milk compared to a non-transgenic littermate. For example, the angiogenin concentration in the milk of a subject transgenic animal, is at least about 2.5%, at least about 5%, at least about 10%, at least about 15%, at least about 20%, at least about 25%, at least about 30%, at least about 35%, at least about 40%, at least about 45%, or at least about 50% higher than the level in a control animal, e.g., a non-transgenic animal of the same species, such as a non-transgenic female littermate. In some embodiments, the level of angiogenin in the milk of a subject transgenic ruminant is about two-fold, about three-fold, about four-fold, or about five-fold higher than in the milk of a control animal.

A transgene having a coding region for angiogenin is used to transform a cell, meaning that a permanent or transient genetic change, generally a permanent genetic change, is induced in a cell following incorporation of the exogenous DNA of the transgene. A permanent genetic change is generally achieved by introduction of the DNA into the genome of the cell. Vectors for stable integration include plasmids, retroviruses and other animal viruses, YACs, and the like.

Transgenic animals of the invention comprise an exogenous nucleic acid sequence present as an extrachromosomal element or stably integrated in all or a portion of its cells, especially in germ cells. Unless otherwise indicated, it will be assumed that a transgenic animal comprises stable changes to the germline sequence. A subject transgenic animal may be heterozygous or homozygous for the transgene. During the initial construction of the animal, “chimeras” or “chimeric animals” are generated in some methods (e.g., where ES cells are used), in which only a subset of cells have the altered genome. Chimeras are primarily used for breeding purposes in order to generate the desired transgenic animal. Animals having a heterozygous alteration are generated by breeding of chimeras. Male and female heterozygotes are typically bred to generate homozygous animals.

In some embodiments, the angiogenin transgene that is introduced into a cell includes an exogenous angiogenin coding sequence. The exogenous gene is in some embodiments from a different species than the animal host (e.g., is a heterologous angiogenin gene). The exogenous gene may or may not be altered in its coding sequence. Non-coding sequences, such as control elements, may or may not be present. Control elements, if present in the transgene, include homologous (e.g., normally associated with the coding sequence) or heterologous (e.g., not normally associated with the coding region, e.g., from another species) control elements. The introduced gene may be a wild-type gene, naturally occurring polymorphism, or a genetically manipulated sequence, for example having deletions, substitutions or insertions in the coding or non-coding regions. The angiogenin coding region may be operably linked to a promoter, which may be constitutive or inducible, and other regulatory sequences required for expression in the host animal. Alternatively, the angiogenin coding region may not be operably linked to a control element(s) in the transgene, but instead becomes operably linked to control element(s) when it becomes integrated into the genome. By “operably linked” is meant that a DNA sequence and a regulatory sequence(s) are connected in such a way as to permit gene expression when the appropriate molecules, e.g. transcriptional activator proteins, are bound to the regulatory sequence(s).

In other embodiments, the endogenous angiogenin coding sequence is upregulated. In these embodiments, the angiogenin coding sequence may or may not be operably linked to control element(s). The angiogenin coding region may be operably linked to a promoter, which may be constitutive or inducible, and other regulatory sequences required for expression in the host animal. Alternatively, the angiogenin coding region may not be operably linked to a control element(s), but instead becomes operably linked to control element(s) when the transgene becomes integrated into the genome. For example gene editing using zinc finger transcription factors fused to endonucleases (Sangamo technology) may be a useful approach for upregulating endogenous angiogenin expression in muscle or mammary gland or liver, or more likely expression systemically.

The angiogenin transgene may comprise angiogenin from any species but particularly includes from human, bovine, porcine, equine, avian, ovine, rat, chicken, turkey or mouse angiogenin. The transgene may encode angiogenin having SEQ ID NO: 1 (human), SEQ ID NO: 2 (bovine), SEQ ID NO: 3 (mouse), SEQ ID NO: 4 (chicken), SEQ ID NO: 5 (rabbit), SEQ ID NO: 6 (pig), SEQ ID NO: 7 (horse), or any other sequence encoding angiogenin or a functional fragment thereof capable of inducing growth of myoblasts in cell culture.

```
(SEQ ID NO: 1)
                10 20 30 40 50 60
MVMGLGVLLL VFVLGLGLTP PTLAQDNSRY THFLTQHYDA KPQGRDDRYC ESIMRRRGLT
                70 80 90 100 110 120
SPCKDINTFI HGNKRSIKAI CENKNGNPHR ENLRISKSSF QVTTCKLHGG SPWPPCQYRA
              130 140
TAGFRNVVVA CENGLPVHLD QSIFRRP
(SEQ ID NO: 2)
                10 20 30 40 50 60
MVMVLSPLLL VFILGLGLTP VAPAQDDYRY IHFLTQHYDA KPKGRNDEYC FNMMKNRRLT
                70 80 90 100 110 120
RPCKDRNTFI HGNKNDIKAI CEDRNGQPYR GDLRISKSEF QITICKHKGG SSRPPCRYGA
              130 140
TEDSRVIVVG CENGLPVHFD ESFITPRH
(SEQ ID NO: 3)
                10 20 30 40 50 60
MAISPGPLFL IFVLGLVVIP PTLAQDDSRY TKFLTQHHDA KPKGRDDRYC ERMMKRRSLT
                70 80 90 100 110 120
SPCKDVNTFI HGNKSNIKAI CGANGSPYRE NLRMSKSPFQ VTTCKHTGGS PRPPCQYRAS
              130 140
AGFRHVVIAC ENGLPVHFDE SFFSL
(SEQ ID NO: 4)
                10 20 30 40 50 60
MAMSSLWWTA ILLLALTVSM CYGVPTYQDF LRTHVDFPKT SFPNIAAYCN VMMVRRGINV
                70 80 90 100 110 120
HGRCKSLNTF VHTDPRNLNT LCINQPNRAL RTTQQQLPVT DCKLIRSHPT CSYTGNQFNH
              130
RVRVGCWGGL PVHLDGTFP
(SEQ ID NO: 5)
                10 20 30 40 50 60
QDDSRYKHFL TQHYDAKPFG RNDRYCETMM KRRDLTSPCK DTNTFVHGNK GSIKDVCEDK
                70 80 90 100 110 120
NGKPYGKNFR ISKSSFQVTT CKHVGGSPWP PCRYRATSGS RNIVIACENG LPVHFDESVF
QQKVH
(SEQ ID NO: 6)
                10 20 30 40 50 60
KDEDRYTHFL TQHYDAKPKG RDGRYCESIM KQRGLTRPCK EVNTFIHGTR NDIKAICNDK
                70 80 90 100 110 120
NGEPYNNFRR SKSPFQITTC KHKGGSNRPP CGYRATAGFR TIAVACENGL PVHFDESFII
TSQ
(SEQ ID NO: 7)
                10 20 30 40 50 60
MAMSLCPLLL VFVLGLGLTP PSLAQDDSRY RQFLTKHYDA NPRGRNDRYC ESMMVRRHLT
                70 80 90 100 110 120
TPCKDTNTFI HGSKSSIKAI CGNKNGNPYG ETLRISKTRF QVTTCKHAGG SPRPPCRYRA
              130 140
TPGFRSIVIA CENGLPVHFD ESFFRP
```

The transgenic animals of the present invention are non-human, including, but not limited to farm animals (pigs, goats, sheep, cows, horses (also known as ungulates or hooved animals, and including ruminants)), rodents (such as rats, guinea pigs and mice), poultry (e.g., avian species such as chickens, ducks, geese, turkeys, etc.), and lagomorphs (e.g., rabbits). Livestock animals such as pigs, sheep, goats and cows, (e.g., ungulates and ruminants), as well as poultry, are of particular interest.

In one embodiment the animal comprises a natural variant with a spontaneous mutation or a natural variant with upregulated angiogenin expression. Where the animal is bovine and angiogenin is introduced to increase muscle mass the animal may include beef cattle breeds in which double muscled animals are known to occur at high to moderate frequency, for example Belgium Blue, Blonde d'Aquitaine, Charolais, Gasconne, Limousin, Maine-Anjou, Parthenaise, Asturiano, Rubia Gallega, Piedmontese, Angus and Hereford.

Where the animal is bovine and the angiogenin is introduced to produce angiogenin in milk at increased concentration the animal may include cattle breeds that are highly specialised for milk production, such as Friesian and Jersey Cattle.

Such cattle could be selectively bred to overexpress angiogenin without reliance on the transgenes of the present invention and thus provide a non-transgenic alternative for increased meat production or increased angiogenin concentration in milk.

Skeletal muscle specific promoters are known to persons skilled in the art and direct expression of the angiogenin transgene to muscle cells. Promoters capable of directing expression of a transgene to muscle cells include dystrophin promoter, a mef2c promoter (Heidt, A and Black, B (2005) Genesis 42:28-32), or promoters using the muscle specific enhancer MEN1. Synthetic muscle promoters may also be used, such as described in WO 2004/041177. An actin promoter can be used for more general expression to tissues such as the liver, which would have a similar effect on muscle. The casein promoter may be useful for beef animals due to an early muscle development advantage from extra angiogenin in the milk.

Mammary gland specific promoters will be apparent to the skilled artisan and include, for example, a F-casein gene promoter (e.g., comprising a sequence set forth in SEQ ID NO: 8) or a prolactin-inducible mammary specific promoter (e.g., comprising a sequence set forth in SEQ ID NO: 9) or a R-lactalbumin gene promoter (e.g., comprising a sequence set forth in SEQ ID NO: 10) or a whey acidic protein (WAP) gene promoter (e.g., comprising a sequence set forth in SEQ ID NO: 11 or 12 or 13) or a F-lactoglobulin gene promoter (e.g., comprising a sequence set forth in SEQ ID NO: 14 or 15 or 16. Each of these promoters confers expression on a nucleic acid operably linked thereto at least in a mammary epithelial cell at least during lactation. A lactalbumin promoter also confers expression on a nucleic acid linked thereto in at least mammary epithelial cells during pregnancy.

| (SEQ ID NO: 8) | |
|---|---|
| tcgaatccat ctctatcaat taatgtaatt caaaattggt gagagacagt cattaggaaa | 60 |
| ttctctgttt attgcacaat atgtaaagca tcttcctgag aaaagggaaa tgttgaatgg | 120 |
| gaaggacatg ctttcttttg tattcctttt ctcagaaatc acactttttt gcctgtggcc | 180 |
| ttggcaacca aaagctaaca cataaagaaa ggcatatgaa gtagccaagg ccttttctag | 240 |
| ttatatctat gacactgagt tcatttcatc atttattttc ctgacttcct cctgggccat | 300 |
| atgagcagtc ttagaatgaa tattagctga ataatccaaa tgcatagtag atgttgattt | 360 |
| gggttttcta agcaatacaa gacttctatg acagtgagat gtattaccat ccaacacaca | 420 |
| tctcagcatg atataaatgt aaggtatatt gtgaagaaaa attatcaatt atgtcaaagt | 480 |
| gcttacttta gaagatcatc tatctgtccc aaagctgtga atatatatat tgaacataat | 540 |
| taatagacga aacaaacctt gtaaaaatga gtagtgtaaa atacaactac atttatgaac | 600 |
| atctatcact aaagaggcaa agaaagttga ggactgcttt tgtaaatggg ctcttattaa | 660 |
| tgaaaagtac ttttgaggtc tggcttagac tctattgtag tacttatggt aagaccctcc | 720 |
| tcttgtctgg gctttcattt tctttcttcc ttccctcatt tgcccttcca tgaatactag | 780 |
| ctgataaaca ttgactcact ataaaagata tgaggccaaa cttgagctgt ccattttaat | 840 |
| aaatctgtat aaataatatt tgttctacag aagtatctct aaataaatgt actttctctc | 900 |
| ttaaaatccc tcaacaaatc cccactatct agagaataag attgacattc cctggagtca | 960 |
| cagcatgctt tgtctgccat tatctgaccc ctttctcttt ctctcttctc acctccatct | 1020 |
| actccttttt ccttgcaata catgacccag attcactgtt tgatttggct tgcatgtgtg | 1080 |
| tgtgctgagt tgtgtctcac tcttgtcaac cccatgaatg acagtccacc aggctccact | 1140 |
| atttccagtt aagaatactg gagtggattg tgtttcctac ttcatttgat taatttagtg | 1200 |
| actttttaaa tttttttcca tattcaggag gctattcttt ccttttagtc tatactgtct | 1260 |
| tcgctcttca ggtctaagct atcatcatgt gcttgttagc ttgtttcttt ctccattata | 1320 |
| gcataaacac taacaactat tcaggttagc atgagattgt gttctttgtg tggcctgtgt | 1380 |
| atttctggtg tgtattagaa tttaccccaa gatctcaaag acccaccgaa tactaaagag | 1440 |
| acctcattgt agttacaata atttggggac tgggccaaaa cttccgtgtg tcccagccaa | 1500 |
| ggtctgtagc tactggacaa tttaatttcc tttatcagat tgtgaattat tccctttaaa | 1560 |
| atgctcccca gaatttttgg ggacagaaaa ataggaagaa ttcattttct aatcatgcag | 1620 |
| atttctagga attcaaatcc actattggtt ttatttcaaa ccacaaaatt agcatgccat | 1680 |
| taaatactat atataaacaa ccacaaaatc agatcattat cca | 1723 |
| (SEQ ID NO: 9) | |
| ggatccaagt agtagttgag tctcatgcta aatgccacca tgttccatcc cttttcccaa | 60 |
| ggctctcagt tatgagtctc catatcaagg ggctttcctg gactttgtcc tatggctagg | 120 |
| ttggacagac aaatatcacc tttgatccta ggatgtgata catccccttt ccacgttctg | 180 |
| tatgtgttta ggggtaagca tggagttggc tgtagccaac tgtgttttcc agtcacctcc | 240 |
| cttgtattgt ctctgaagcc tcctttgttc caaaagtagg ttaaggaaat cctgcttcct | 300 |
| ggaagcagcc ctaaaagaaa tgaaggttta ccagagccaa gtgagaagct gggtcatgtg | 360 |
| tggaattatg tgggaagaaa acaatacttg gtattgactg gatcgaggag atggggggag | 420 |
| ggtggcagga tggagggagg ctggcaggct cagggtttct attttggcat aagcatctct | 480 |
| tcatcattgt cttcctagag agaaggcccg gtgccaggag gccagaggcc ttcttcatac | 540 |
| ataaaagcag atgaagtgag cggtgtctgc attacaaggt ccaggagcag tcaaa | 595 |
| (SEQ ID No: 10) | |
| tatttagtgg tattggtggt tggggatggg gaagctgata acatctcaga gggtagctag | 60 |
| atactgtcat acacactttt caagttctcc atttttgtga aatagaaagt ctctggatcc | 120 |
| aagttatatg tgattctcag tctctgtggt catattctat ttctattcct gaccactcaa | 180 |
| caaggaacca agatatcaag ggacacttct tttgtttcat gcctgggttg agtgggccag | 240 |
| tgtcagctct gatcctggga ccatgacata cgatgatgta cagtcctttc ccatattctg | 300 |
| tatgtctcta aggggaagga ggagttggcc atggaccctt tgtgcatttt ctcattttct | 360 |
| gattgcttca cttgtattac ccctgaggcc ccctttgttc ctgaagtatg ttgggcacat | 420 |
| cttgcttcct agaaccaaca ctaccagaaa caacataaag ccaaatggga aacaggatca | 480 |
| tgtttgtaac actgcttggg caggtaacaa tacctagtat ggactagaga ttctggggag | 540 |
| gaaaggaaaa gtggggtgaa attactgaag gaagctagca ggctcaatgt ttctttgttg | 600 |
| gttttactgg cctctctcgt catcctcttc ctggatgtaa ggcttgatgc cagggcccct | 660 |
| gaggcttttt ccacaaataa aaggaggtga gcagtgtggt gacccc | 706 |
| (SEQ ID NO: 11) | |
| gaattctttc actgctaaaa cagggcggga ggagtccaga gccctgccac tgggtgcaga | 60 |
| acatgaagac cccttgagtg gaaaggggtt atacagctgg acagtggtgg cgcacacctt | 120 |
| taatcccagc actcgggagg cagaggcaga cggatctctg agttcgaggc cagcctggtc | 180 |
| tacagaatga gttccgggac agccagggcc acacagagaa actcttgtct cgaaaaacca | 240 |
| aaaaaaaaca aaaaaggaaa ggggttacac aacagagact caggtcacag ctacccatca | 300 |
| cacacaggat acacatacaa aggtgttcac aggcagatga ggaacgagga gaaggggctc | 360 |
| aagcaagggc ctaaagtttc tttttttttt tttcttcttt ttttttttcc ctgtggccta | 420 |
| gagtttcaag aggctgagga cctaggcatg aaccaagagg ggccaaacca cttcaagaag | 480 |
| cagggggtag cagcagaatc tcactatcag ccttgagcac agctgggaag gagatccatg | 540 |
| gaaacaacca agaaagagct gaaaggggct ggagagatgg ctcagcagtt aagagcactg | 600 |
| agtgctcttc cgaagtccta gattcaaatc ccagcaacca tatggtgggt cacaaccatc | 660 |
| tgtaatgaga tctgatgccc tcttctggtg tgtctgaaga cagctacagt gtacttatgt | 720 |
| ataataaata aataaatctt tagaaaggga gggggggaga gagagagaga gagagagaga | 780 |
| gagagagaga gagagagaga gagagagaga gagctggaag agggagatct gggaagtctg | 840 |
| ctggctttat atgctgacca tatatagtca cctgtgttta cacactgtgc tcatcacttt | 900 |
| gaaatctcag tggtttcttc tttgagcctg tgtctgtaag ttcaccagga gagtggtaca | 960 |
| taggcaagaa taacagccag tgggcatagg acacagagtg catgggcccc agcaagactg | 1020 |
| tagagagaac agagctctgg ctcctaagac atagggcctt ctgggaaact caagcagcca | 1080 |
| agcaacccta gccagccctt tcctggtggc cctccttctg ttccagcaaa ggcggaaatg | 1140 |
| ggaacagggg tggaagcaga gcattggcag agcataggta tgacttagtc ttgactaaca | 1200 |
| caagcatggc agtagcctga cagtggccta aatgtgggga tgactgcctt agatggggat | 1260 |
| gactgcctta gatgggcatg actgccttag atggggatga ctgccttaga tggggatgac | 1320 |
| tgccttagat ggaacaacaa acatctatgg gcatgctgtg gaacactggc ccacacacgg | 1380 |
| aactgaaggc actggcaatt tccatagggc agttaaacct aaaagcatgc tcacactcaa | 1440 |
| caggctgccg gaatctcatg agacacctgg aatagacgaa tgtagaaaca gagcagagag | 1500 |
| ttggttgcca aggtctgggg gctcagagga caagcaagag gcgcggcttt cctttggggc | 1560 |
| tggcatgaaa ggaaatatcg aggttacagc ctgagagggc ttcccctgac acttcgtatt | 1620 |
| caaagaggcc atgggcacca gtgaagacaa aggagtatgg cctgcaccac aggctggcnc | 1680 |
| tgacagtcag taagcacaca gtcactctgg gtcatcccat ccccttcctt gcaagagaaa | 1740 |
| tcaaggaaat gtcccgagaa caatggggca cagtgccagc aggacatctc ttcctgccca | 1800 |
| tggcaccctt tggcacggta tgggcccttc tgggaaggtg gccttccaaa ttgctctgca | 1860 |
| caggcagctc cttttcaatg tatgcccgac actctctaca tggagcaagc gcctccacac | 1920 |
| tcttagaaga attttagaaa actccagaaa agcaccagga gaagtcaccc tcagatgtag | 1980 |
| cccggactcg agccttgctc aaaacctcct gtcttgtttt ctatgtgacc tgtacaaatt | 2040 |
| tggagctcag aattgccttt gtctgtgatg ggttccaacc caaccactca aagtgacact | 2100 |
| tgtcacattt gtcactgatc ctatttcttc tttttctgct ccttcatttt ctccgctttc | 2160 |
| ataataaaca agtattactt tttaagtggg ggaaaaaatg accaccctta caaaggactt | 2220 |
| tttaaaaatg gcctccattg tggcccttgt tcctggcagc ctgggcctgc tctctctgtg | 2280 |
| tggccaagaa ggaagtgttg tagcccatct agagctgtgc cagcctcttc ccccacccca | 2340 |
| cccccaaagt cttcctcctg tgggtccttt aaatgcatcc cagacactca gacagccatc | 2400 |
| agtcacttgc | 2410 |
| (SEQ ID NO: 12) | |
| tctgaagagg ggacattttg tgacctgcca acatgcaaag ttaccaaaac atagcaagtc | 60 |
| gccatcggcc aggacctcta gaccccagtc gctaaagctc agtgctggct acccagggag | 120 |
| gggcctggac tgaggtccta gaactctgct gaggccttgt agggactgag atggtgctac | 180 |
| ctggggcctg gggcctgggg cctggggcct ggagcggggt gagccagggg gaccgtagca | 240 |
| gcctgtcaaa gtggaagggt gttctgggca tctggaactg catgcagtcc aggctgaggg | 300 |
| ccccagagaa gtactgaggg gctctgtgtc caaggccaag aagccacagg ccaggcagag | 360 |
| gagtggggcc tggaccaggg gtgggcactg accaccagca cacgcagtca tcccgggcac | 420 |
| accttccttg tccaagccct cagggcaaaa ggatcaagga aattccccgg agaaggaggg | 480 |
| cacctagcct gagtgatcat cctgtcccca ccccggtccc tacacaggga cacaggcggg | 540 |
| gcccttctgg gaataggctt tcccagtgtc tgccctgcgc agaaacagcc ccgaccctga | 600 |
| acctgcctgc ccctcccttt ctaagacgcc cgacatcctc tgcacagagc atacggctcc | 660 |
| taagtacaag acgactcgtt cttgccgtgg aaagttcggg aaaagacaaa aagatactgc | 720 |
| aggaggaata aaatgccctc caaggtcccc ccacacccgg gctcctcctc cttctcctct | 780 |
| cccggcggac ggagtgctga tttggacacg tcactccctg tccctgaagg gcctctccac | 840 |
| gaccactgtg ttgtgtcatt gcggattcta tttcttcctt tgtctgccct taattttttg | 900 |
| aatgttcaca ataaacatgc attactttca aagtggaaaa aaatggatcc actttatgag | 960 |
| gaattctttt ttatttaaaa acgtggccca aggcagtggc cgcccagtct ggggtcggtc | 1020 |
| caagctggaa gtcttttggt ccaactgggg cagggccagc cactcaccct cccccaccgt | 1080 |
| gttcttcttc ccgcctcctc ctttaaaggt gccccagggc cacgagccac catctgtcac | 1140 |
| ctgcctgcca cctgccacca tgcgctgtct tgccagctct ggcctctggc ctctgatctc | 1200 |
| ctctggaggc tgctctttgc actggcccca gccatctcct tgccaggtaa gcccaggagg | 1260 |
| ggcatcctgc catccctctg ctccaggtcc ccccaccccc cgaaatgctg cccaggcctc | 1320 |
| acagtttggt ga | 1332 |
| (SEQ ID NO: 13) | |
| aggaaagcac actcgacact cgaacggact gcctactgtc agatcccatt tacatgagat | 60 |
| gcccagaata gacagacgca gaaaccgagc agagaggtag ttgccaaggc ctgggggctc | 120 |
| ggggaactag cgagaggctg ctggcaggca caggttttcc tttggggctg gcctgaaacg | 180 |
| aaacatcaag gttacagcct gaaagagctt cccctgggac tttgtcttca aagaggagag | 240 |
| gccatgggcc acagtgaaga cctccggcca gtcaaaggag tatgggctgc accataggct | 300 |
| ggcgcgacag ccagtaaaca cacagtcact cactctcgag tcattgcatc cccttccttg | 360 |
| caagagaagt caaggaaatg tcccgagagc aatgggcaca gtgcccaaca ggacatccca | 420 |
| tccgggccca tgacaccgtt ggcacagcat ggggcccttc tgagaagtgg gctttcaagg | 480 |
| ttccctgcac aggcaatcct tttttgatgt gtaccctgta ctctctacaa ggagcaagtg | 540 |
| cctccacatt cttataaaac tttttagaaa actccagaaa agcaccaaga aaagaaacca | 600 |
| tcctctgatg tgactgtaca catttggagc tcggaatttc cttttttttt tttttttaaa | 660 |
| gatttttatt tatttcatgt atgggagcac actgtcgcta tcttcagaca caccagaaga | 720 |
| gggcatcaga tcccactgga tcccagatgg ttgtgagcca ccatgtggtt gctgggacct | 780 |
| gaactcagga cctctggaag agcagtcagt gctcccaacc actgagccat ctctccagcc | 840 |
| ctcggaattt cctttgtccg agaaaggggt cccaacccaa ccattcaaag tgatatctgt | 900 |
| cacatttgtt acagatccca tttcttcctt ctctgctcct taattttttt cgttttggcc | 960 |
| ataaacaagt tttacctttt aagtgaaaaa ataacgacca cccttacaaa ggacttctta | 1020 |
| aaaatggact ccgaattgtg aaccttgttc tggtagcctg ggcctgctct ctgcatgtgt | 1080 |
| ccaagaggaa gtgttttagc ccatctacgc ctatgcaagc ctgcccccct ccttccccaa | 1140 |
| agtcttcctc ctgtgggtcc tttaaat | 1167 |
| (SEQ ID NO: 14) | |
| ggcccagagg gggacttcct gcttggcccc ggatggaaga aggcctccta ttgtcctcgt | 60 |
| agaggaagcc accccggggc ccggggatga gccaagtagg attccgggaa cctcgtggct | 120 |
| ggggcgcggc ccgggctggc tggctggcac gcctcctgta taaggccccg agcccgctgt | 180 |
| ctcagccctc cactc | 195 |
| (SEQ ID NO: 15) | |
| gtcgacctgc aggtcaacgg atctctgtgt ctgttttcat gttagtacca cactgttttg | 60 |
| gtggctgtag ctttcagcta cagtctgaag tcataaagcc tggtacctcc agctctgttc | 120 |
| tctctcaaga ttgtgttctg ctgtttgggt ctttagtgtc tccacacaat ttttagaatt | 180 |
| gtttgttcta gttctgtgaa aaatgatgct ggtattttga taaggattgc attgaatctg | 240 |
| taaagctaca gatatagtca ttgggtagta cagtcacttt aacaatatta actcttcaca | 300 |
| tctgtgagca tgatatattt tccccctcta tatcatcttc aattcctcct atcagtttct | 360 |
| ttcattgcag ttttctgagt acaggtctta cacctccttg gttagagtca ttcctcagta | 420 |
| ttttattcct ttgatacaat tgtgaatgag gtaattttct tagtttctct ttctgatagc | 480 |
| tcattgttag tgtatatata gaaaagcaac agatttctat gtattaattt tgtatcctgc | 540 |
| aacagatttc tatgtattaa ttttgtatcc tgctacttta cggaattcac ttattagctt | 600 |
| tttggtgaca tcttgaggat tttctgaaga aaatggcatg gtatggtagg acaaggtgtc | 660 |
| atgtcatctg caaacagtgg cagttttcct tcttcccttc caacctggat ttctttgatt | 720 |
| tctttctgtc tgagtacgac taggattccc aatactatac cgaataaaag tggcaagagt | 780 |
| ggacatcctt gtcttatttt tctgacctta gaggaaatgc tttcagtttt tcaccattaa | 840 |
| ttataatgtt tactgtgggc ttgtcatatg tggccttcat tatatggagg tctattccct | 900 |
| ctatacccac cttgttgaga gtttttatca taaaagtatg ttgaattttg tcaaaagttt | 960 |
| ttcctgcatc tattgagatg atttttactc ttcaattcat taatgatttt tattcttcat | 1020 |
| tttgttaatg atttccattc ttcaatttgt taacgtggta tatcacattg attgatttgt | 1080 |
| ggataccttt gtatccctgg gataaacctc acttgatcat gagctttcaa tgtatttttg | 1140 |
| aattcacttt gctaatattc tgttgggtat ttttgcatct ctattcatca atgatattgg | 1200 |
| cctaagaaag gttttgtctg gttttagtat cagggtgatg ctggcctcat agagagagtt | 1260 |
| tagaagcatt tcctcctctt tgatttttcg gaatagtttg agtaggatag gtattaactc | 1320 |
| ttctttaaat gtttggggac ttccctggtg agccggtggt tgagaatccg cctcagggat | 1380 |
| gtgggtttga tccctggtca gggaaccatt aataagatcc cacatgctgc aggcaacaag | 1440 |
| cccccaagct gcaaccactg agctgcaacc gctgcagtgc ccacaggcca cgaccagaga | 1500 |
| aagcccacat acagcaggga agacccagca caaccggaaa aaggagtttg gtggaataca | 1560 |
| gctgtgaagc cgtctggtcc tggactcctg cttgagggaa ttttttaaaa attattgatt | 1620 |
| caatttcatt actggtaact ggtctgttca tattttctat ttcttccggg ttcagtcttg | 1680 |
| ggagattgta catgcctagg aatgtgtccg tttcttctag gttgtccatt ttattggaca | 1740 |
| tgcatgggag cacacagcac cgaccagcga gactcatgct ggcttcctgg ggccaggctg | 1800 |
| gggccccaag cagcatggca tcctagagtg tgtgaaagcc cactgaccct gcccagcccc | 1860 |
| acaatttcat tctgagaagt gattccttgc ttctgcactt acaggcccag gatctgacct | 1920 |
| gcttctgagg agcaggggtt ttggcaggac ggggagatgc tgagagccga cgggggtcca | 1980 |
| ggtcccctcc caggcccccc tgtctggggc agcccttggg aaagattgcc ccagtctccc | 2040 |
| tcctacagtg gtcagtccca gctgccccag gccagagctg ctttatttcc gtctctctct | 2100 |
| ctggatggta ttctctggaa gctgaaggtt cctgaagtta tgaatagctt tcgggtgaag | 2160 |
| ggcatggttt gtggtcacgg ttcacaggaa gctcgggaga ccctgcagct cagacgtccc | 2220 |
| gagattggtg gcacccagat ttcctaagcC cgctggggaa cagggcgctt gtttctccct | 2280 |
| ggctgacctc cctcctccct gcatcaccca gttctgaaag cagagcggtg ctggggtcac | 2340 |
| agcctctcgc atctaacgcc ggtgtccaaa ccacccgtgc tggtgttcgg ggggctacct | 2400 |
| atggggaagg gcttctcact gcagtggtgc cccccgtccc ctctgagatc agaagtccca | 2460 |
| gtccggacgt caaacaggcc gagctccctc cagaggctcc agggagggat ccttgccccc | 2520 |
| ccgctgctgc ctccagctcc tggtgccgca cccttgagcc tgatcttgta gacgcctcag | 2580 |
| tctagtctct gcctccgtgt tcacacgcct tctccccatg tcccctccgt gtccccgttt | 2640 |
| tctctcacaa ggacaccgga cattagatta gcccctgttc cagcctcacc tgaacagctc | 2700 |
| acatctgtaa agacctagat tccaaacaag attccaacct gaagttcccg gtggatgtga | 2760 |
| gttctggggc gacatccttc aaccccatca cagcttgcag ttcatcgcaa aacatggaac | 2820 |
| ctggggttta tcgtaaaacc caggttcttc atgaaacact gagcttcgag gcttgttgca | 2880 |
| agaattaaag gtgctaatac agatcagggc aaggactgaa gctggctaag cctcctcttt | 2940 |
| ccatcacagg aaaggggggc ctgggggcgg ctggaggtct gctcccgtga gcgagctctt | 3000 |
| tcctgctaca gtcaccaaca gtctctctgg gaaggaaacc agaggccaga gagcaagccg | 3060 |
| gagctagttt aggagacccc tgaacctcca cccaagatgc tgaccagcca gcgggccccc | 3120 |
| tggaaagacc ctacagttca ggggggaaga ggggctgacc cgccaggtcc ctgctatcag | 3180 |
| gagacatccc cgctatcagg agattccccc accctgctcc cgttccccta tcccaatacg | 3240 |
| cccaccccac ccctgtgatg agcagtttag tcacttagaa tgtcaactga aggcttttgc | 3300 |
| atcccctttg ccagaggcac aaggcaccca cagcctgctg ggtaccgacg cccatgtgga | 3360 |
| ttcagccagg aggcctgtcc tgcaccctcc ctgctcgggc cccctctgtg ctcagcaaca | 3420 |
| cacccagcac cagcattccc gctgctcctg aggtctgcag gcagctcgct gtagcctgag | 3480 |
| cggtgtggag ggaagtgtcc tgggagattc aaaatgtgag aggcgggagg tgggaggttg | 3540 |
| gqccctgtgg gcctgcccat cccacgtgcc tgcattagcc ccagtgctgc tcagccgtgc | 3600 |
| ccccgccgca ggggtcaggt cactttcccg tcctggggtt attatgactc ttgtcattgc | 3660 |
| cattgccatt tttgctaccc taactgggca gcaggtgctt gcagagccct cgataccgac | 3720 |
| caggtcctcc ctcggagctc gacctgaacc ccatgtcacc cttgccccag cctgcagagg | 3780 |
| gtgggtgact gcagagatcc cttcacccaa ggccacggtc acatggtttg gaggagctgg | 3840 |
| tgcccaaggc agaggccacc ctccaggaca cacctgtccc cagtgctggc tctgacctgt | 3900 |
| ccttgtctaa gaggctgacc ccggaagtgt tcctggcact ggcagccagc ctggacccag | 3960 |
| agtccagaca cccacctgtg cccccgcttc tggggtctac caggaaccgc ctaggcccag | 4020 |
| aggggacttc ctgcttggcc ttggatggaa gaaggcctcc tattgtcctc gtagaggaag | 4080 |
| ccaccccggg gcctgaggat gagccaagtg ggattccggg aaccgcgtgg ctgggggccc | 4140 |
| agcccgggct ggctggcctg catgcctcct gtataaggcc ccaagcctgc tgtctcagcc | 4200 |
| ctcc | 4204 |
| (SEQ ID NO: 16) | |
| gtcaacggat ctctgtgtct gttttcatgt tagtaccata ctgttttggt ggctgtagct | 60 |
| ttgagctata gtctgaagtc ataaagcccg atacctccag ctctgttctt ctttctcaag | 120 |
| attgtgttct gctgtttggg tctttagtgt ctccacacaa tttttagaat tgtgtgttct | 180 |
| agttctgtga aaaatgatgc tggcattttg ataaggattg cattgaatct gtaaagctac | 240 |
| agatatagtc attgggtagt acaatcactt taacaatatt aactcttcaa atccgtgagc | 300 |
| atgatgtatt ttccccctcc atatcatctt caattccttc tatcagtttc tttcattgca | 360 |
| gttttctgag tataggtctt acacctcctt gattagagtc attcctcagt attttattcc | 420 |
| tttgatacaa ttgtgaatga gatcattttc ttagtttctc tttctgatag cccattgtta | 480 |
| gtgtatagaa aagcaacaga gttctatgta ttaattttgt atcctgcaac agatttctat | 540 |
| gtattaattt tgtatcctgc tactttactg aatttactta ttagcttttt ggtgacatct | 600 |
| taaggatttt cttaagaaaa tggcatggta tggtaggaca aggtgtcacg tcatctgcaa | 660 |
| acagtggcag ttttacttct tcccttccag cctggatttc tttgatttct ttctgtctga | 720 |
| gtactgtgac taggattccc aatactatac cgaacaaaag tggcaagagt ggacatcctt | 780 |
| gtcttatttt tctgacctta gaggaaatgc tttcagtttt tcaccattaa ttataatgtt | 840 |
| tactgtgggc ttgtcatatg tggccttcat tatatggagg tctattccct ctatacccac | 900 |
| tttgttgaga gtttttatca tgaaagtatg ttgaattttg tcaacagttt ttcctgcatc | 960 |
| tattgagatg atttttactc ttcaattcat taatgatttt tattcttcat tttgttaatg | 1020 |
| atttccattc ttcaatgtgt taacgtggta tatcacattg attgatttgt ggatatcttt | 1080 |
| gtatccctgg gataaacctc acctgatcat gagctttcaa tgtatttttg aattcacttt | 1140 |
| gctaatattc tgctgggtat ttttacatct ctattcatca atgatattga cctaagattt | 1200 |
| tctttctttt tttttttgta aagtttttgt gtggttttag tatcagggtg atgctggcct | 1260 |
| catagagaga gtttagaagc atttcctcct ctttgatttt ttggaatagt ttgagtagga | 1320 |
| taggtattaa ctcttcttta aatgtttggg gacttccctg gtgagccggt ggttgagaat | 1380 |
| ccgcctcagg gatgtgggtt tgatccctgg tcagggaacc attaataagc tcccacatgc | 1440 |
| tgcagggcaa caagccccca agctgcaacc actgagctgc aaccgctgca gtgcccacgg | 1500 |
| gccacgacca gagaaagccc acatacagca gggaagaccc agcacaacct aaaaaaggag | 1560 |
| tttggtggaa tacagctgtg aagccatctg gtcctggact cctgcttgag ggaatttttt | 1620 |
| taaaattatt gattcaattt cattactgat tgccccagtc tccctcccac agtggtcagt | 1680 |
| cccagctgcc ccaggccaga ggtgctttat ttccgtctct ctctctggat ggtattcttt | 1740 |
| ggaagctgaa gattcctgga agttatgaat agcttcgccc tgaagggcat ggtttatggt | 1800 |
| cacggttcac aggaacttgg gagaccctgc agctcagacg tcccgaggtt ggtggcaccc | 1860 |
| agatttccta agctcgctgg ggaagggggc gcttgtttct ccctggctga cctccctccg | 1920 |
| ccctgcatca cccagttctg agagcagagc ggtgctgggg ggcacagcct ctcgcatctg | 1980 |
| acgccggtgt ccaaaccacc cgtgctggtg ttcggggggc tacctatggg gaagggctcc | 2040 |
| tcactgcagg ggtgcccccc gtcccctctg agatcagaag tcccagtccg gacagcgaac | 2100 |
| aggccaagct ccctccagag gctccaggaa gggatccttg ccccccgccg ccgcctccag | 2160 |
| ctcctggtgc cgcacccttg agcctgatct tgtagacgcc tcagtctagt ctctgcctcc | 2220 |
| gtgttcacat gccttctccc catgtcccct ccatgtcccc gttttctctc acaaggacac | 2280 |
| cggacagtag attagcccct gttccagcct cacctgaaca gctcacatct gtaaagacct | 2340 |
| agattccaaa caagattcca acctgaagtt cctggtggat gtgagttctg gggcaacatc | 2400 |
| cttcaacccc atcacagctt gcagttcatc acaaaacatg gaacctgggg tttatcataa | 2460 |
| aacctaggtt cttcatgaaa cactgagctt cgaggcttgt tgcaagaatt aaaggtgcta | 2520 |
| atacagatca aggcaaggac tgaagctggc caagcctact ctttccatca caggaaaggg | 2580 |
| gggtctgggg gcggctgggg gtctgctccc gtgagtgagc tcttttctgc tacagtcacc | 2640 |
| aacagtctct ccgggaagga aaccagaggc cagagagcaa gccagagcta gtttaggaga | 2700 |
| cccccgaacc tccaaccaag atgctgacca ggccagcggg ccccctggaa agaccctaca | 2760 |
| gttcaggggg gaagaggggc tgacccgcca ggtccctgct atcaggagac atccccgcta | 2820 |
| tcaggagatt cccccacctt gctcccgttc cgctacccca atacgcccac cccacccctg | 2880 |
| tgatgagtgg tttagccact tagaatgtca actgaaggct tttgcaccct ctttgccaga | 2940 |
| ggcacaaggc acccacagcc cgctgggtac caacgcccat gtggattcag ccaggaggcc | 3000 |
| tgtcctgcac cctccctgct cgggcccctt ctgtactcag caacacaccc agcaccagca | 3060 |
| ttcccactgc tcctgaggtc tgcaggcagc tcgctgtagc ctgagcggtg tggagggaag | 3120 |
| tgtcctggga gacttaaaat gtgggaggtg ggaggggggg aggttgggcc ctgtgggcct | 3180 |
| gcccaccccg tgtgcctgca tggagcccca gtgctgctca gccgtgcccc cgccgcaggg | 3240 |
| gtcaggtcac tttcccgtcc tgggggttat tatgaccgtt gtcattttca ttgccatttt | 3300 |
| tgctacccta actgggcagc aggtgcttgc agagccctcg ataccgacca ggtcccccct | 3360 |
| cggagctcca cctgaacccc gtgtcaccct tgccccagcc tgcagaggat ggggtcactg | 3420 |
| cagagatccc ttcacccaag gccacggtca catggtttgg aggagctggt gcccaaggca | 3480 |
| gaggccaccc tctaggacac acctgtcccc agtgctggct ctgacctgcc cttgtctaag | 3540 |
| aggctgaccc cggaagtgtt cctggcactg gcagccagcc tgacccagag tccagacacc | 3600 |
| cacctgtgcc cccacttctg gggtctacca ggaaccgtct aggcccagag ggggacttcc | 3660 |
| tgcttggccc cggatggaag aaggcctcct attgtcctcg tagaggaagc caccccgggg | 3720 |
| cccggggatg agccaagtag gattccggga acctcgtggc tgggggcccg gcccgggctg | 3780 |
| gctggctggc acgcctcctg tataaggccc cgagcccgct gtctcagccc tccg | 3834. |

### Methods of Making a Subject Transgenic Animal

The invention provides methods of generating a subject transgenic animal. The method generally involves introducing an angiogenin transgene, into a pluripotent or totipotent cell such that the transgene is integrated into the genome of the cell, and transferring the cell into an oviduct of a synchronized recipient female of the same species as the cell.

In some embodiments, a subject transgenic animal is produced by introducing into a single cell embryo a polynucleotide that comprises a nucleotide sequence that encodes angiogenin or fragments or variants thereof, in a manner such that the polynucleotide is stably integrated into the DNA of germ line cells of the mature animal, and is inherited in normal Mendelian fashion. In accordance with the invention, a polynucleotide can be introduced into an embryo by a variety of means to produce transgenic animals. For instance, totipotent or pluripotent stem cells, zygotes (fertilized oocytes), embryonic cells, or somatic cells can be transformed by microinjection, calcium phosphate mediated precipitation, liposome fusion, electroporation, retroviral infection or by other means. Where the transformed cell is other than a zygote or embryonic cell, the transformed cells can then be introduced into embryos and incorporated therein to form transgenic animals.

In many embodiments, a polynucleotide is injected into an embryo, e.g., at the single-cell stage, forming a genetically modified embryo, and the genetically modified embryo is allowed to develop into a mature transgenic animal.

In some embodiments, the transgene is introduced into a somatic cell, where the transgene is integrated into the genome, forming a genetically modified somatic cell, and the nucleus of the genetically modified somatic cell is transferred into a single-cell embryo, forming a genetically modified embryo. The genetically modified single-cell embryo is then transferred into an oviduct of a recipient female, and the embryo allowed to develop into a mature transgenic animal.

Any method of making transgenic animals can be used as described, as will be well known to persons skilled in the art.

Transgenic animals also can be generated using methods of nuclear transfer or cloning using embryonic or adult cell lines as described in the art. Cytoplasmic injection of DNA can be used. Subject transgenic animals can be obtained by introducing a construct comprising angiogenin encoding sequences.

Transgenic animals also include somatic transgenic animals, e.g., transgenic animals that include a transgene in somatic cells (and not in germ line cells). For example, the mammary gland cells of an animal are transformed with an angiogenin transgene, and the transgene is expressed in mammary cells of the animal. Methods of somatic cell transformation are described in the art.

Methods for making transgenic goats are known in the art. See, e.g., Zou et al. (2002) Mol. Reprod. Dev. 61:164-172; Baldassare et al. (2002) Theriogenol. 57:275-284; and Ko et al. (2000) Transgenic Res. 9:215-222. Methods for making transgenic goats are also described in the Examples. Methods for making transgenic cows are known in the art, and are described in, e.g., van Berkel et al. (2002) Nat. Biotechnol. 20:484-487.

Methods for making transgenic pigs are known in the art. See, e.g., U.S. Pat. Nos. 6,344,596; 6,262,336; and 6,218,596.

Methods for making transgenic chickens are known in the art. See, e.g., Harvey et al. (2002) Nat. Biotechnol. 20:396-399; Takami et al. (2002) Biochem. Biophys. Res. Comm. 292:88-93; Harvey et al. (2002) Poultry Sci 81:202-212. Generally, the method involves introducing a stearoyl CoA transgene into a chicken embryo, where the transgene is in an avian retroviral construct, or other suitable construct.

### Expression Vectors and Transgenes

A subject transgenic animal is typically generated by a method involving introducing into a cell a construct comprising a nucleotide sequence encoding angiogenin. An angiogenin transgene includes, at a minimum, a coding region for angiogenin. In some embodiments, the nucleotide sequence encoding angiogenin is operably linked to a promoter and, optionally, additional control elements, that provide for tissue-specific expression of the transgene in the animal. In other embodiments, the nucleotide sequence encoding angiogenin is not operably linked to any control elements. Instead, the angiogenin transgene includes, on the 5′ and 3′ ends of the coding region, sequences that provide for homologous recombination with an endogenous gene.

As discussed above any angiogenin gene can be used in the transgene, including those encoding the angiogenin sequences provided as SEQ ID NO: 1 to 7. The transgene or transgenic animal may also comprise recombinant follistatin.

Sequences that vary from a known coding sequence for a given angiogenin can be used, as long as the encoded angiogenin has substantially the same activity in inducing growth of myoblasts in cell culture. For example, the encoded angiogenin can include one or more conservative amino acid substitutions compared to the amino acid sequence of a known angiogenin. Non-limiting examples of conservative amino acid substitutions are Phe/Tyr; Ala/Val; Leu/Ile; Arg/His; Ser/Thr; etc. The encoded angiogenin can also include insertions or deletions (including truncations) of one or more amino acid residues, compared to the amino acid sequence of a known angiogenin. Further, the encoded angiogenin can include one or more naturally occurring polymorphisms. The angiogenin coding sequence can be completely or partially synthetic. An angiogenin coding sequence can also be a consensus sequence, derived, e.g., by comparing the angiogenin coding sequences from two or more species, and deriving therefrom a consensus sequence, using standard methods. An optimised angiogenin sequence can also be used, for example a sequence that includes mutations that confer greater activity, more protease resistance, etc.

The angiogenin expression cassette or transgene may include a fragment and, or variant of the naturally occurring angiogenin gene. Certain fragments include one or more conserved domains such as sequences encoding a catalytic core or a cell binding site. By a “catalytic core” is meant an internal region of the polypeptide excluding signal peptide and N- and C-terminal variable regions.

Two distinct regions of human angiogenin are required for its angiogenic activity including a catalytic site containing His-13, Lys-41, and His-115 that is capable of cleaving RNA and a noncatalytic, cell binding site encompassing minimally residues 60-68. RNase activity and receptor binding capacity, while required, are not sufficient for angiogenic activity: endocytosis and nuclear translocation are required as well.

Activity may be increased or decreased by changing key amino acids at or near the active site, with improved activity by substituting Asp-116 with H is being an example. Functional studies indicate Arg-5 and Arg-33 are also important for activity.

Cellular uptake of angiogenin in proliferating endothelial cells is mediated by domains and is not dependent upon RNase activity as enzymatically inactive mutants can be internalized. K41Q and H13A mutants for example are enzymatically inactive but are translocated. Improved versions of angiogenin more readily internalised by cells and more potent are within the scope of the present invention, and such variants can be tested for by conducting in vitro uptake and activity tests on epithelial and muscle cells in culture.

Any known coding sequence for angiogenin can be used as a base to make a subject transgenic animal, including an angiogenin coding sequence from mouse, human, cow, sheep, etc. The coding sequence can be a cDNA sequence, or a genomic sequence. The coding sequence for the angiogenin may be, but need not be, from the same species as the transgenic animal.

A suitable nucleotide sequence encoding angiogenin generally has at least about 70%, at least about 80%, at least about 85%, at least about 90%, at least about 95%, or at least about 98%, or higher, nucleotide sequence identity with a known coding sequence for angiogenin. Sequence similarity is calculated based on a reference sequence, which may be a subset of a larger sequence, such as a conserved motif, coding region, flanking region, etc. A reference sequence will usually be at least about 18 nucleotides long, more usually at least about 30 nucleotides long, and may extend to the complete sequence that is being compared. Algorithms for sequence analysis are known in the art, such as BLAST, described in Altschul et al. (1990), J. Mol. Biol. 215:403-10 (using default settings).

Also suitable for use are angiogenin coding sequences that hybridize under stringent hybridization conditions to a known angiogenin coding sequence. An example of stringent hybridization conditions is hybridization at 50° C. or higher and 0.1×SSC (15 mM sodium chloride/1.5 mM sodium citrate). Another example of stringent hybridization conditions is overnight incubation at 42° C. in a solution: 50% formamide, 1×SSC (150 mM NaCl, 15 mM sodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C. For example, high stringency conditions include aqueous hybridization (e.g., free of formamide) in 6×SSC (where 20×SSC contains 3.0 M NaCl and 0.3 M sodium citrate), 1% sodium dodecyl sulfate (SDS) at 65° C. for about 8 hours (or more), followed by one or more washes in 0.2×SSC, 0.1% SDS at 65° C. For example, moderate stringency conditions include aqueous hybridization (e.g., free of formamide) in 6×SSC, 1% SDS at 65° C. for about 8 hours (or more), followed by one or more washes in 2×SSC, 0.1% SDS at room temperature.

As noted above, in some embodiments, an angiogenin transgene includes a coding sequence for angiogenin operably linked to one or more control sequences, e.g., promoters, 3′ transcriptional control sequences, translational control elements, etc.

In many embodiments, an angiogenin transgene includes a coding region for angiogenin operably linked to one or more tissue-specific control elements, e.g., a tissue-specific promoter, and optionally additional tissue-specific control elements (e.g., a 3′ untranslated region, an enhancer, and the like). The tissue-specific control element(s) can be heterologous, e.g., not normally operably linked to an angiogenin coding sequence in nature, or homologous, e.g., normally operably linked to an angiogenin coding sequence in nature. Tissue-specific control elements provide for expression of the angiogenin transgene preferentially in a given tissue, e.g., such control elements are more active (e.g., 2-fold, 5-fold, 10-fold, 20-fold, or 50-fold more active, or greater than 50-fold more active) in a given tissue than in other tissues under normal physiological conditions. A wide variety of tissue-specific promoters are known in the art.

Promoters useful for production of angiogenin in the milk of a subject transgenic animal are active in mammary tissue, e.g., the promoters are more active in mammary tissue than in other tissues under physiological conditions in which milk is synthesized. Suitable promoters provide for both specific and efficient transcription in mammary tissue. Mammary gland-specific promoters are strong promoters in mammary tissue that can support the synthesis of large amounts of protein for secretion into milk. Mammary gland-specific promoters include, but are not limited to, a whey acidic protein (WAP) promoter; αS1 casein, αS2 casein, β casein, and kappa casein promoters; an α-lactalbumin promoter; a lactoferrin promoter; and a β-lactoglobulin (“BLG”) promoter. The sequences of a number of mammary gland-specific promoters have been isolated and their nucleotide sequences have been published and some are provided as SEQ ID NO: 8 to 16.

Suitable intestinal epithelial cell-specific promoters include, but are not limited to, a T3(b) gene promoter; a villin gene promoter; a keratin 19 gene promoter; a calbindin-D9K gene promoter; a lactase gene promoter; and an intestinal fatty acid binding protein promoter; and the like.

Skeletal muscle specific promoters are known to persons skilled in the art and direct expression of the angiogenin transgene to muscle cells. Promoters capable of directing expression of a transgene to muscle cells include dystrophin promoter, a mef2c promoter (Heidt, A and Black, B (2005) Genesis 42:28-32), or promoters using the muscle specific enhancer MEN1. Synthetic muscle promoters may also be used, such as described in WO 2004/041177. An actin promoter can be used for more general expression to tissues such as the liver, which would have a similar effect on muscle. The casein promoter may be useful for beef animals due to an early muscle development advantage from extra angiogenin in the milk.

Where the transgenic animal expresses the angiogenin transgene in all tissues, a strong constitutive, or an inducible promoter, is used. Strong constitutive promoters include, but are not limited to, strong promoters active in eukaryotic cells, including a promoter from cytomegalovirus (CMV), mouse mammary tumor virus (MMTV), Rous sarcoma virus (RSV), or adenovirus. Exemplary promoters include the promoter from the immediate early gene of human CMV; the promoter from the long terminal repeat (LTR) of RSV; SV40 early promoter; and the adenovirus major late promoter. Such promoters are of particular interest where the transgenic animal is a transgenic poultry.

In some embodiments, an angiogenin transgene is not operably linked to a control element. Instead, the transgene includes sequences that provide for homologous recombination with an endogenous gene, such that the angiogenin coding sequence replaces all or part of endogenous coding sequence, and the integrated angiogenin coding region is under transcriptional control of endogenous control element(s). For example, an angiogenin transgene includes 5′ and 3′ flanking sequences that are homologous to sequences in the 5′ and 3′ regions of a β-lactoglobulin gene, such that the transgene integrates into the genome of a cell by homologous recombination, whereby the angiogenin coding sequences of the transgene replace the endogenous β-lactoglobulin gene, and the angiogenin coding sequence integrates into the genome and is under the transcriptional control of the endogenous β-lactoglobulin control elements. Methods for carrying out homologous recombination are well known in the art.

An angiogenin transgene is generally provided as part of a vector (e.g., an angiogenin construct), a wide variety of which are known in the art and need not be elaborated upon herein. Vectors include, but are not limited to, plasmids; cosmids; viral vectors; artificial chromosomes (HACs, YACs, BACs, etc.); mini-chromosomes; and the like. Vectors are amply described in numerous publications well known to those in the art. Vectors provide for expression of the subject nucleic acids, may provide for propagating the subject nucleic acids, or both.

For expression, e.g., where the transgene includes a promoter, an expression cassette may be employed. The expression vector will provide a transcriptional and translational initiation region, which may be inducible or constitutive, where the coding region is operably linked under the transcriptional control of the transcriptional initiation region, and a transcriptional and translational termination region. These control regions may be native to a gene encoding the subject peptides, or may be derived from exogenous sources.

Where the transgene includes a promoter, an expression vector will generally have convenient restriction sites located near the promoter sequence to provide for the insertion of nucleic acid sequences encoding angiogenin. A selectable marker operative in the expression host may be present. Expression vectors may be used for the production of fusion proteins, where the exogenous fusion peptide provides additional functionality, i.e. increased protein synthesis, stability, protein secretion, reactivity with defined antisera, an enzyme marker, e.g. β-galactosidase, etc.

Expression cassettes may be prepared comprising a transcription initiation region, the gene or fragment thereof, and a transcriptional termination region.

### Utility

The subject transgenic animals find use in a variety of applications, including, but not limited to, food production, research, production of angiogenin and the like. For example, the subject animals find use in producing food products that have higher angiogenin or greater muscle development than those produced naturally. Such food products can be used as a source of angiogenin. The subject animals find use in research, to analyze the effects of angiogenin and its proposed modulators in various tissues.

### Food Applications

The present invention provides methods for producing food products from a subject transgenic animal, and food products harvested from a subject transgenic animal. The methods generally involve harvesting a food product from a subject transgenic animal. Where the food product requires further processing, the methods involve harvesting a food product from a subject transgenic animal, and processing the food product. Thus, the invention provides a method of producing a processed food product, involving processing a food product harvested from a subject transgenic animal. The invention further provides a processed food product obtained by processing a food product harvested from a subject transgenic animal.

Methods of harvesting food products from a subject transgenic animal are well known to those in the agricultural and food production industries. Where a subject transgenic animal expresses the angiogenin transgene in milk, the milk is harvested by the usual means. Where a subject transgenic animal expresses the angiogenin transgene in epithelial cells of the intestine and/or rumen, meat (muscle tissue) is harvested by standard abattoir methods. Where the subject transgenic animal is a transgenic poultry, and the food product is an egg, eggs are harvested in the usual manner. Methods of processing a food product harvested from a subject transgenic animal are standard in the food processing art and are well known to those in the field.

The present invention further provides food products produced by a subject transgenic animal, and processed food products made with such food products. A subject food product includes a food product that contains a meat, egg, or milk product of a subject transgenic animal. Food products include any preparation for human consumption including for enteral or parenteral consumption, which when taken into the body (a) serve to nourish or build up tissues or supply energy and/or (b) maintain, restore or support adequate nutritional status or metabolic function.

Where a subject transgenic animal expresses the angiogenin transgene in milk, food products include milk, and any food products made from or containing milk, including, but not limited to, cheese, yogurt, butter, ice cream, and other frozen desserts, whipped toppings, cream, custard, pudding, nutritional drinks, infant formula, and chocolate.

Where a subject transgenic animal expresses the angiogenin transgene in epithelial cells of the intestine and/or rumen, food products include meat, meat products, and food products containing meat. Meats include beef, veal, pork, mutton, lamb, goat meat, and the like. Meat products include processed meats such as bologna, sausages, salami, ham, bacon, and the like.

Where a subject transgenic animal is a poultry animal, food products include eggs, and food products made from or containing eggs or a portion of an egg (e.g., egg yolk, egg white); and poultry meat.

Food products of the invention are suitable for consumption by any individual. As used herein, the term “individual” includes human and non-human individuals. Non-human individuals include animals, particularly mammals, e.g., farm animals such as cows, pigs, sheep, goats and poultry, pets and companion animals such as horses, cats, dogs, guinea pigs, rats and mice, and aquatic animals such as fish and animals used for aquaculture etc. The transgenic non-human animal can be used as animal feed in appropriate circumstances.

A variety of beneficial effects are attributed to angiogenin, including increased muscle function and mass, improved fat to weight composition, exercise tolerance, and involvement in neuro-muscular disease such as ALS.

Angiogenin can also increase the growth rate of farm animals fed with a diet that includes angiogenin. Thus, a subject food product increases the growth rate and feed efficiency of a farm animal fed with a subject food product. Thus, a subject food product is of particular interest for feeding a farm animal (e.g., a pig, a cow, a sheep, a goat, etc.).

The present invention provides food products, including nutraceutical formulations, that include angiogenin. The term “nutraceutical formulation” refers to a food or part of a food that offers medical and/or health benefits including prevention or treatment of disease. Nutraceutical products range from isolated nutrients, dietary supplements and diets, to genetically engineered designer foods, functional foods, herbal products and processed foods such as cereal, soup and beverages. The term “functional foods,” refers to foods that include “any modified food or food ingredients that may provide a health benefit beyond the traditional nutrients it contains.”

Nutraceutical formulations of interest include foods for veterinary or human use, including food bars (e.g. cereal bars, breakfast bars, energy bars, nutritional bars); chewing gums; drinks; fortified drinks; drink supplements (e.g., powders to be added to a drink); tablets; and the like.

A subject food product or nutraceutical formulation includes angiogenin and at least one additional food-grade component. Suitable components include, but are not limited to, mono- and disaccharides; carbohydrates; proteins; amino acids; fatty acids; lipids; stabilizers; preservatives; flavoring agents; coloring agents; sweeteners; antioxidants, chelators, and carriers; texturants; nutrients; pH adjusters; emulsifiers; stabilizers; milk base solids; edible fibers; and the like. The food component can be isolated from a natural source, or can be synthesized. All components are food-grade components fit for human consumption.

Examples of suitable monosaccharides include sorbitol, mannitol, erythrose, threose, ribose, arabinose, xylose, ribulose, glucose, galactose, mannose, fructose, and sorbose. Non-limiting examples of suitable disaccharides include sucrose, maltose, lactitol, maltitol, maltulose, and lactose.

Suitable carbohydrates include oligosaccharides, polysaccharides, and/or carbohydrate derivatives. As used herein, the term “oligosaccharide” refers to a digestible linear molecule having from 3 to 9 monosaccharide units, wherein the units are covalently connected via glycosidic bonds. As used herein, the term “polysaccharide” refers to a digestible (i.e., capable of metabolism by the human body) macromolecule having greater than 9 monosaccharide units, wherein the units are covalently connected via glycosidic bonds. The polysaccharides may be linear chains or branched. Carbohydrate derivatives, such as a polyhydric alcohol (e.g., glycerol), may also be utilized as a complex carbohydrate herein. As used herein, the term “digestible” in the context of carbohydrates refers to carbohydrate that are capable of metabolism by enzymes produced by the human body. Examples of polysaccharides that are non-digestible carbohydrates are cellulose, resistant starches (e.g., raw corn starches) and retrograded amyloses (e.g., high amylose corn starches). Non-limiting examples carbohydrates include raffinoses, stachyoses, maltotrioses, maltotetraoses, glycogens, amyloses, amylopectins, polydextroses, and maltodextrins.

Suitable fats include, but are not limited to, triglycerides, including short-chain (C₂-C₄) and long-chain triglycerides (C₁₆-C₂₂).

Suitable texturants (also referred to as soluble fibers) include, but are not limited to, pectin (high ester, low ester); carrageenan; alginate (e.g., alginic acid, sodium alginate, potassium alginate, calcium alginate); guar gum; locust bean gum; psyllium; xanthan gum; gum arabic; fructo-oligosaccharides; inulin; agar; and functional blends of two or more of the foregoing.

Suitable emulsifiers include, but are not limited to, propylene glycol monostearate (PGMS), sodium stearoyl lactylate (SSL), calcium stearoyl lactylate (CSL), monoglycerides, diglycerides, monodiglycerides, polyglycerol esters, lactic acid esters, polysorbate, sucrose esters, etc.

Edible fibers include polysaccharides, oligosaccharides, lignin and associated plant substances. Suitable edible fibers include, but are not limited to, sugar beet fiber, apple fiber, pea fiber, wheat fiber, oat fiber, barley fiber, rye fiber, rice fiber, potato fiber, tomato fiber, other plant non-starch polysaccharide fiber, and combinations thereof.

Suitable flavoring agents include natural and synthetic flavors, “brown flavorings” (e.g., coffee, tea); dairy flavorings; fruit flavors; vanilla flavoring; essences; extracts; oleoresins; juice and drink concentrates; flavor building blocks (e.g., delta lactones, ketones); and the like; and combinations of such flavors. Examples of botanic flavors include, for example, tea (e.g., preferably black and green tea), aloe vera, guarana, ginseng, ginkgo, hawthorn, hibiscus, rose hips, chamomile, peppermint, fennel, ginger, licorice, lotus seed, schizandra, saw palmetto, sarsaparilla, safflower, St. John's Wort, curcuma, cardamom, nutmeg, cassia bark, buchu, cinnamon, jasmine, haw, chrysanthemum, water chestnut, sugar cane, lychee, bamboo shoots, vanilla, coffee, and the like.

Suitable sweeteners include, but are not limited to, alitame; dextrose; fructose; lactilol; polydextrose; xylitol; xylose; aspartame, saccharine, cyclamates, acesulfame K, L-aspartyl-L-phenylalanine lower alkyl ester sweeteners, L-aspartyl-D-alanine amides; L-aspartyl-D-serine amides; L-aspartyl-hydroxymethyl alkane amide sweeteners; L-aspartyl-1-hydroxyethylalkane amide sweeteners; and the like.

Suitable anti-oxidants include, but are not limited to, tocopherols (natural, synthetic); ascorbyl palmitate; gallates; butylated hydroxyanisole (BHA); butylated hydroxytoluene (BHT); tert-butyl hydroquinone (TBHQ); and the like.

Suitable nutrients include vitamins and minerals, including, but not limited to, niacin, thiamin, folic acid, pantothenic acid, biotin, vitamin A, vitamin C, vitamin B₂, vitamin B₃, vitamin B₆, vitamin B₁₂, vitamin D, vitamin E, vitamin K, iron, zinc, copper, calcium, phosphorous, iodine, chromium, molybdenum, and fluoride.

Suitable coloring agents include, but are not limited to, FD&C dyes (e.g., yellow #5, blue #2, red #40), FD&C lakes; Riboflavin; β-carotene; natural coloring agents, including, for example, fruit, vegetable, and/or plant extracts such as grape, black currant, aronia, carrot, beetroot, red cabbage, and hibiscus.

Exemplary preservatives include sorbate, benzoate, and polyphosphate preservatives.

Suitable emulsifiers include, but are not limited to, diglycerides; monoglycerides; acetic acid esters of mono- and diglycerides; diacetyl tartaric acid esters of mono- and diglycerides; citric acid esters of mono- and diglycerides; lactic acid esters of mono- and diglycerides; fatty acids; polyglycerol esters of fatty acids; propylene glycol esters of fatty acids; sorbitan monostearates; sorbitan tristearates; sodium stearoyl lactylates; calcium stearoyl lactylates; and the like.

Suitable agents for pH adjustment include organic as well as inorganic edible acids. The acids can be present in their undissociated form or, alternatively, as their respective salts, for example, potassium or sodium hydrogen phosphate, potassium or sodium dihydrogen phosphate salts. Exemplary acids are edible organic acids which include citric acid, malic acid, fumaric acid, adipic acid, phosphoric acid, gluconic acid, tartaric acid, ascorbic acid, acetic acid, phosphoric acid and mixtures thereof.

Angiogenin is present in the food product/nutraceutical formulation in an amount of from about 0.01% to about 50% by weight, e.g., from about 0.01% to about 0.1%, from about 0.1% to about 0.5%, from about 0.5% to about 1.0%, from about 1.0% to about 2.0%, from about 2.0% to about 5%, from about 5% to about 7%, from about 7% to about 10%, from about 10% to about 15%, from about 15% to about 20%, from about 20% to about 25%, from about 25% to about 30%, from about 30% to about 35%, from about 35% to about 40%, from about 40% to about 45%, or from about 45% to about 50% by weight.

Where the food product is a beverage, the food product generally contains, by volume, more than about 50% water, e.g., from about 50% to about 60%, from about 60% to about 95% water, e.g., from about 60% to about 70%, from about 70% to about 80%, from about 80% to about 90%, or from about 90% to about 95% water.

Where the food product is a bar, the food product generally contains, by volume, less than about 15% water, e.g., from about 2% to about 5%, from about 5% to about 7%, from about 7% to about 10%, from about 10% to about 12%, or from about 12% to about 15% water.

In some embodiments, the food product/nutraceutical is essentially dry, e.g., comprises less than about 5%, water.

Monosaccharides, disaccharides, and complex carbohydrates, if present, are generally present in an amount of from about 0.1% to about 15%, e.g., from about 0.1% to about 1%, from about 1% to about 5%, from about 5% to about 7%, from about 7% to about 10%, or from about 10% to about 15%, by weight each. Soluble fibers, edible fibers, and emulsifiers, if present, are generally present in an amount of from about 0.1% to about 15%, e.g., from about 0.1% to about 1%, from about 1% to about 5%, from about 5% to about 7%, from about 7% to about 10%, or from about 10% to about 15%, by weight each.

Other components discussed above, if present, are present in amounts ranging from about 0.001% to about 5% by weight of the composition.

### Research Applications

The subject transgenic animals find use in research, to analyze the effects of angiogenin and its proposed modulators in various tissues. The subject transgenic animals are useful for studying the regulation of muscle synthesis. In particular, the subject transgenic animals are useful for studying the regulation of transcription and translation of angiogenin.

### Application as a Source of Angiogenin

The subject transgenic animals may be used as a source of angiogenin, particularly those animals that express angiogenin in milk. The angiogenin may be isolated from the milk by techniques known to those skilled in the art. For example, cation exchange purification (or enrichment), or size selection may be used.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Example 1

### Generation and Characterization of Transgenic Mice and Cows

### Materials and Methods

### Constructs

A DNA construct designed to express the bovine angiogenin cDNA in the mammary gland under control of the milk protein gene beta-lactoglobulin (Big) promoter is made by standard DNA cloning techniques.

Both transgenic mice and cows are produced with the DNA construct by standard pronuclear microinjection procedures. Briefly, donor females are superovulated, bred and one-cell fertilized zygotes are collected from the oviducts. Zygotes with visible pronuclei are microinjected with approximately 2 picolitres of the DNA construct at a concentration of 5 ng/μl and surviving zygotes are surgically transferred into the oviducts of synchronized recipient females. Pregnancies are carried to term and all offspring born are screened for the presence of the transgene with construct-specific polymerase chain reaction (PCR) primers. PCR-positive founders are confirmed by Southern blot.

Primary transgenic mice and cows are bred to non-transgenic control mice and cows, respectively, of the same genetic background. Non-transgenic female siblings of the F1 and F2 transgenic animals were used as controls.

### Results

Milk samples are collected once per mouse per lactation at peak lactation (days 10-12) and analyzed for angiogenin using standard methods well known in the art.

Reverse transcriptase PCR analysis of mRNA from transgenic angiogenin and non-transgenic control cows is carried out. Mammary gland tissue from transgenic and non-transgenic control lactating cows is collected by biopsy. Total RNA is isolated from the tissue by standard procedures, quantified and used for reverse transcriptase PCR. 1 μg of total RNA is treated with DNaseI for 1 hour at 37° C. After heat denaturation, MMLV reverse transcriptase is added and samples incubated at 40° C. for one hour followed by heat inactivation. 1 μl of each reverse transcriptase reaction is then used for PCR.

### Example 2

### Transgenic Animals Expressing an Angiogenin Transgene in Intestinal

### Epithelial Cells

Two DNA constructs are made, which promote expression of the human angiogenin the small intestine of the mouse under the control of the rat FABPi gene. A 1.2 kb fragment of the rat FABPi promoter from −1179 to +28 is included in the DNA construct. This region of promoter has been shown to promote tissue-specific expression of the human growth hormone gene at levels similar to the endogenous FABP gene. The FABPi promoter region is amplified from mouse genomic DNA using a polymerase chain reaction (PCR) with forward primer 5′GAATTCCTTAATTTGCATAA3′ (SEQ ID NO: 01) from −1179 and reverse primer 5′CTCGAGCAGCTGTGTCATAGTTCT3′ (SEQ ID NO:02) from +28. The resulting fragment has an XhoI restriction enzyme site on the 3′ end and is cloned into the pGemTEasy vector (Promega). The same human angiogenin cDNA used in Example I is then inserted in the above vector. Orientation of the fragment is determined by PCR and verified by sequencing.

Transgenic mice are generated by standard pronuclear microinjection procedures as routinely performed in our laboratory. Briefly, C57B1/6×CBA females are superovulated at 3-4 weeks of age and bred to intact males. One-cell zygotes are collected, microinjected and surgically transferred to synchronous CD1 recipient females. Resulting pups are identified as transgenic by PCR of toe clips taken at 10 days of age. Transgene-specific primers spanning the junction of the FABPi and angiogenin are used. The presence of the transgene is confirmed by Southern blotting with a probe spanning the two sequences. Founder animals are bred to non-transgenic control mice of the same background to establish lines. The level of angiogenin RNA is analyzed by Northern blot analysis.

The Examples provided above demonstrate that the present invention provides transgenic non-human animals expressing an angiogenin transgene in milk or the muscle, and that the milk from such animals has a greater angiogenin composition than milk from non-transgenic littermate and that the muscle tissue from such animals has a higher degree of muscle hyperplasia than muscle tissue from a non-transgenic littermate.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A non-human transgenic animal comprising a transgene encoding angiogenin.

2. The transgenic non-human animal of claim 1, wherein said animal is a mammal.

3. The transgenic non-human animal of claim 2, wherein said mammal is an ungulate.

4. The transgenic non-human animal of claim 1, wherein said animal is poultry.

5. The transgenic non-human animal of claim 1, wherein said transgene is chromosomally integrated.

6. The transgenic non-human animal of claim 1, wherein said transgene comprises a coding sequence for angiogenin operably linked to an animal tissue specific promoter.

7. The transgenic non-human animal of claim 6, wherein said animal tissue specific promoter is a mammary specific promoter.

8. The transgenic non-human animal of claim 6, wherein said animal tissue specific promoter is a muscle specific promoter.

9. An expression cassette comprising a coding sequence for angiogenin operably linked to a heterologous mammalian tissue-specific promoter.

10. The expression cassette of claim 9, wherein said heterologous tissue specific promoter is a mammary specific promoter.

11. The expression cassette of claim 9, wherein said heterologous tissue specific promoter is a muscle specific promoter.

12. The expression cassette of claim 9, wherein said expression cassette is present in a vector.

13. A method for producing a non-human transgenic animal comprising an angiogenin transgene, said method comprising:
(a) introducing an angiogenin transgene into a single-celled embryo, forming a genetically modified embryo; and
(b) transferring the genetically modified embryo into a recipient female of the same species as the embryo, wherein the genetically modified embryo develops into a transgenic animal in the female.

14. The method of claim 13, wherein said transgenic animal is chosen from a mouse, a rat, a rabbit, a pig, a sheep, a goat, poultry, and a cow.

15. The method of claim 13, wherein the transgenic animal is a mammal, and said transgene is expressed in mammary gland cells of said mammal.

16. The method of claim 13, wherein the transgenic animal is a mammal, and wherein said transgene is expressed in muscle cells of said mammal.

17. A method for producing a non-human transgenic animal comprising an angiogenin transgene, said method comprising:
a) introducing an angiogenin transgene into a somatic cell, forming a genetically modified somatic cell comprising a genetically modified nucleus;
b) transferring the genetically modified nucleus from the genetically modified somatic cell into a single-celled embryo, generating a genetically modified single-celled embryo; and
c) transferring the genetically modified single-celled embryo into a recipient female of the same species as the embryo, wherein the genetically modified embryo develops into a transgenic animal in the female.

18. A method of producing a food product, said method comprising harvesting a food product from the non-human transgenic animal of claim 1.

19. A method of producing a food product, the method comprising processing a food product harvested from the non-human transgenic animal of claim 1.

20. A food product harvested from the non-human transgenic animal of claim 1.

21. The food product of claim 20, wherein the food product is processed.

22. The food product of claim 20, wherein said food product is milk.

23. The food product of claim 20, wherein said food product is meat.

24. The food product of claim 20, wherein said food product is an egg.

25. (canceled)

26. A method of treating muscle disorders, muscle wasting disorders, muscular dystrophy, muscular atrophy, sarcopenia, cachexia, improving muscle form by improving muscle strength, mass or exercise tolerance, decreasing fat, improving muscle to fat ratio, treating diseases caused by or involving suboptimal muscle to fat ratio which effect is enhanced by follistatin, treating bone disorders, osteoporosis, improving bone density, treating neurological disorders or diseases affecting the nervous system, and treating motor neurone diseases, ALS, spinal muscular atrophys, inflammation myopathies, dermatomyositis, polymyositis, inclusion body myositis, diseases of the neuromuscular junction, Myasthenia Gravis (MG), Lambert-Eaton Syndrome (LES), Congenital Myasthenic Syndrome (CMS), myopathies due to endocrine abnormalities, Hyperthyroid Myopathy (HYPTM), Hypothyroid Myopathy (HYPOTM), diseases of peripheral nerve, Charcot-Marie-Tooth Disease (CMT), Dejerine-Sottas Disease (DS), Friedreich's Ataxia (FA), other myopathies, Myotonia Congenita (MC), Paramyotonia Congenita (PC), Central Core Disease (CCD), Nemaline Myopathy (NM), Myotubular Myopathy (MTM or MM), Periodic Paralysis (PP), metabolic diseases of muscle, Phosphorylase Deficiency (MPD or PYGM), Acid Maltase Deficiency (AMD), Phosphofructokinase Deficiency (PFKM), Debrancher Enzyme Deficiency (DBD), Mitochondrial Myopathy (MITO), Carnitine Deficiency (CD), Carnitine Palmityl Transferase Deficiency (CPT), Phosphoglycerate Kinase Deficiency (PGK), Phosphoglycerate Mutase Deficiency (PGAM or PGAMM), Lactate Dehydrogenase Deficiency (LDHA), Myoadenylate Deaminase Deficiency (MAD), diseases connected to impaired lipid metabolism, dyslipidemia, related lipid abnormalities, hyperlipidemia, hypercholesteremia, hypertriglyceridemia, mixed dyslipidemia, spine injuries or diseases, diseases involving glucose homeostasis, for providing neuroprotection, nervous system functional support, managing metabolic diseases and diseases connected to impaired glucose metabolism and impaired insulin action, diabetes mellitus, diabetes mellitus type 1 and 2, non-autoimmune non-insulin dependent diabetes mellitus, syndrome X or metabolic syndrome, for microbial inhibition, enhancing gut epithelial function, wound healing, and bacterial flora symbiosis, for gut health and gut based disease prevention and immune enhancement by administering to a patient in need thereof a therapeutically effective amount of angiogenin and optionally follistatin derived from milk of the transgenic human animal of claim 7.


SEQUENCE LISTING (18 sequences)

SEQ ID NO: 1 - protein, 147 aa, Homo sapiens
MVMGLGVLLLVFVLGLGLTPPTLAQDNSRYTHFLTQHYDAKPQGRDDRYCESIMRRRGLTSPCKDINTFIHGNKRSIKAICENKNGNPHRENLRISKSSFQVTTCKLHGGSPWPPCQYRATAGFRNVVVACENGLPVHLDQSIFRRP

SEQ ID NO: 2 - protein, 148 aa, Bos taurus
MVMVLSPLLLVFILGLGLTPVAPAQDDYRYIHFLTQHYDAKPKGRNDEYCFNMMKNRRLTRPCKDRNTFIHGNKNDIKAICEDRNGQPYRGDLRISKSEFQITICKHKGGSSRPPCRYGATEDSRVIVVGCENGLPVHFDESFITPRH

SEQ ID NO: 3 - protein, 145 aa, Mus musculus
MAISPGPLFLIFVLGLVVIPPTLAQDDSRYTKFLTQHHDAKPKGRDDRYCERMMKRRSLTSPCKDVNTFIHGNKSNIKAICGANGSPYRENLRMSKSPFQVTTCKHTGGSPRPPCQYRASAGFRHVVIACENGLPVHFDESFFSL

SEQ ID NO: 4 - protein, 139 aa, Gallus gallus
MAMSSLWWTAILLLALTVSMCYGVPTYQDFLRTHVDFPKTSFPNIAAYCNVMMVRRGINVHGRCKSLNTFVHTDPRNLNTLCINQPNRALRTTQQQLPVTDCKLIRSHPTCSYTGNQFNHRVRVGCWGGLPVHLDGTFP

SEQ ID NO: 5 - protein, 125 aa, Oryctolagus cuniculus
QDDSRYKHFLTQHYDAKPFGRNDRYCETMMKRRDLTSPCKDTNTFVHGNKGSIKDVCEDKNGKPYGKNFRISKSSFQVTTCKHVGGSPWPPCRYRATSGSRNIVIACENGLPVHFDESVFQQKVH

SEQ ID NO: 6 - protein, 123 aa, Sus scrofa
KDEDRYTHFLTQHYDAKPKGRDGRYCESIMKQRGLTRPCKEVNTFIHGTRNDIKAICNDKNGEPYNNFRRSKSPFQITTCKHKGGSNRPPCGYRATAGFRTIAVACENGLPVHFDESFIITSQ

SEQ ID NO: 7 - protein, 146 aa, Equus caballus
MAMSLCPLLLVFVLGLGLTPPSLAQDDSRYRQFLTKHYDANPRGRNDRYCESMMVRRHLTTPCKDTNTFIHGSKSSIKAICGNKNGNPYGETLRISKTRFQVTTCKHAGGSPRPPCRYRATPGFRSIVIACENGLPVHFDESFFRP

SEQ ID NO: 8 - DNA, 1723 bp, Artificial Sequence
  F-casein gene promoter
tcgaatccatctctatcaattaatgtaattcaaaattggtgagagacagtcattaggaaattctctgtttattgcacaatatgtaaagcatcttcctgagaaaagggaaatgttgaatgggaaggacatgctttcttttgtattccttttctcagaaatcacacttttttgcctgtggccttggcaaccaaaagctaacacataaagaaaggcatatgaagtagccaaggccttttctagttatatctatgacactgagttcatttcatcatttattttcctgacttcctcctgggccatatgagcagtcttagaatgaatattagctgaataatccaaatgcatagtagatgttgatttgggttttctaagcaatacaagacttctatgacagtgagatgtattaccatccaacacacatctcagcatgatataaatgtaaggtatattgtgaagaaaaattatcaattatgtcaaagtgcttactttagaagatcatctatctgtcccaaagctgtgaatatatatattgaacataattaatagacgaaacaaaccttgtaaaaatgagtagtgtaaaatacaactacatttatgaacatctatcactaaagaggcaaagaaagttgaggactgcttttgtaaatgggctcttattaatgaaaagtacttttgaggtctggcttagactctattgtagtacttatggtaagaccctcctcttgtctgggctttcattttctttcttccttccctcatttgcccttccatgaatactagctgataaacattgactcactataaaagatatgaggccaaacttgagctgtccattttaataaatctgtataaataatatttgttctacagaagtatctctaaataaatgtactttctctcttaaaatccctcaacaaatccccactatctagagaataagattgacattccctggagtcacagcatgctttgtctgccattatctgacccctttctctttctctcttctcacctccatctactcctttttccttgcaatacatgacccagattcactgtttgatttggcttgcatgtgtgtgtgctgagttgtgtctcactcttgtcaaccccatgaatgacagtccaccaggctccactatttccagttaagaatactggagtggattgtgtttcctacttcatttgattaatttagtgactttttaaatttttttccatattcaggaggctattctttccttttagtctatactgtcttcgctcttcaggtctaagctatcatcatgtgcttgttagcttgtttctttctccattatagcataaacactaacaactattcaggttagcatgagattgtgttctttgtgtggcctgtgtatttctggtgtgtattagaatttaccccaagatctcaaagacccaccgaatactaaagagacctcattgtagttacaataatttggggactgggccaaaacttccgtgtgtcccagccaaggtctgtagctactggacaatttaatttcctttatcagattgtgaattattccctttaaaatgctccccagaatttttggggacagaaaaataggaagaattcattttctaatcatgcagatttctaggaattcaaatccactattggttttatttcaaaccacaaaattagcatgccattaaatactatatataaacaaccacaaaatcagatcattatcca

SEQ ID NO: 9 - DNA, 595 bp, Artificial Sequence
  prolactin-inducible mammary specific promoter
ggatccaagtagtagttgagtctcatgctaaatgccaccatgttccatcccttttcccaaggctctcagttatgagtctccatatcaaggggctttcctggactttgtcctatggctaggttggacagacaaatatcacctttgatcctaggatgtgatacatcccctttccacgttctgtatgtgtttaggggtaagcatggagttggctgtagccaactgtgttttccagtcacctcccttgtattgtctctgaagcctcctttgttccaaaagtaggttaaggaaatcctgcttcctggaagcagccctaaaagaaatgaaggtttaccagagccaagtgagaagctgggtcatgtgtggaattatgtgggaagaaaacaatacttggtattgactggatcgaggagatggggggagggtggcaggatggagggaggctggcaggctcagggtttctattttggcataagcatctcttcatcattgtcttcctagagagaaggcccggtgccaggaggccagaggccttcttcatacataaaagcagatgaagtgagcggtgtctgcattacaaggtccaggagcagtcaaa

SEQ ID NO: 10 - DNA, 706 bp, Artificial Sequence
  R-lactalbumin gene promoter
tatttagtggtattggtggttggggatggggaagctgataacatctcagagggtagctagatactgtcatacacacttttcaagttctccatttttgtgaaatagaaagtctctggatccaagttatatgtgattctcagtctctgtggtcatattctatttctattcctgaccactcaacaaggaaccaagatatcaagggacacttcttttgtttcatgcctgggttgagtgggccagtgtcagctctgatcctgggaccatgacatacgatgatgtacagtcctttcccatattctgtatgtctctaaggggaaggaggagttggccatggaccctttgtgcattttctcattttctgattgcttcacttgtattacccctgaggccccctttgttcctgaagtatgttgggcacatcttgcttcctagaaccaacactaccagaaacaacataaagccaaatgggaaacaggatcatgtttgtaacactgcttgggcaggtaacaatacctagtatggactagagattctggggaggaaaggaaaagtggggtgaaattactgaaggaagctagcaggctcaatgtttctttgttggttttactggcctctctcgtcatcctcttcctggatgtaaggcttgatgccagggcccctgaggctttttccacaaataaaaggaggtgagcagtgtggtgacccc

SEQ ID NO: 11 - DNA, 2410 bp, Artificial Sequence
  whey acidic protein gene promoter
gaattctttcactgctaaaacagggcgggaggagtccagagccctgccactgggtgcagaacatgaagaccccttgagtggaaaggggttatacagctggacagtggtggcgcacacctttaatcccagcactcgggaggcagaggcagacggatctctgagttcgaggccagcctggtctacagaatgagttccgggacagccagggccacacagagaaactcttgtctcgaaaaaccaaaaaaaaacaaaaaaggaaaggggttacacaacagagactcaggtcacagctacccatcacacacaggatacacatacaaaggtgttcacaggcagatgaggaacgaggagaaggggctcaagcaagggcctaaagtttctttttttttttttcttctttttttttttccctgtggcctagagtttcaagaggctgaggacctaggcatgaaccaagaggggccaaaccacttcaagaagcagggggtagcagcagaatctcactatcagccttgagcacagctgggaaggagatccatggaaacaaccaagaaagagctgaaaggggctggagagatggctcagcagttaagagcactgagtgctcttccgaagtcctagattcaaatcccagcaaccatatggtgggtcacaaccatctgtaatgagatctgatgccctcttctggtgtgtctgaagacagctacagtgtacttatgtataataaataaataaatctttagaaagggagggggggagagagagagagagagagagagagagagagagagagagagagagagagagagagagctggaagagggagatctgggaagtctgctggctttatatgctgaccatatatagtcacctgtgtttacacactgtgctcatcactttgaaatctcagtggtttcttctttgagcctgtgtctgtaagttcaccaggagagtggtacataggcaagaataacagccagtgggcataggacacagagtgcatgggccccagcaagactgtagagagaacagagctctggctcctaagacatagggccttctgggaaactcaagcagccaagcaaccctagccagccctttcctggtggccctccttctgttccagcaaaggcggaaatgggaacaggggtggaagcagagcattggcagagcataggtatgacttagtcttgactaacacaagcatggcagtagcctgacagtggcctaaatgtggggatgactgccttagatggggatgactgccttagatgggcatgactgccttagatggggatgactgccttagatggggatgactgccttagatggaacaacaaacatctatgggcatgctgtggaacactggcccacacacggaactgaaggcactggcaatttccatagggcagttaaacctaaaagcatgctcacactcaacaggctgccggaatctcatgagacacctggaatagacgaatgtagaaacagagcagagagttggttgccaaggtctgggggctcagaggacaagcaagaggcgcggctttcctttggggctggcatgaaaggaaatatcgaggttacagcctgagagggcttcccctgacacttcgtattcaaagaggccatgggcaccagtgaagacaaaggagtatggcctgcaccacaggctggcnctgacagtcagtaagcacacagtcactctgggtcatcccatccccttccttgcaagagaaatcaaggaaatgtcccgagaacaatggggcacagtgccagcaggacatctcttcctgcccatggcaccctttggcacggtatgggcccttctgggaaggtggccttccaaattgctctgcacaggcagctccttttcaatgtatgcccgacactctctacatggagcaagcgcctccacactcttagaagaattttagaaaactccagaaaagcaccaggagaagtcaccctcagatgtagcccggactcgagccttgctcaaaacctcctgtcttgttttctatgtgacctgtacaaatttggagctcagaattgcctttgtctgtgatgggttccaacccaaccactcaaagtgacacttgtcacatttgtcactgatcctatttcttctttttctgctccttcattttctccgctttcataataaacaagtattactttttaagtgggggaaaaaatgaccacccttacaaaggactttttaaaaatggcctccattgtggcccttgttcctggcagcctgggcctgctctctctgtgtggccaagaaggaagtgttgtagcccatctagagctgtgccagcctcttcccccaccccacccccaaagtcttcctcctgtgggtcctttaaatgcatcccagacactcagacagccatcagtcacttgc

SEQ ID NO: 12 - DNA, 1332 bp, Artificial Sequence
  whey acidic protein gene promoter
tctgaagaggggacattttgtgacctgccaacatgcaaagttaccaaaacatagcaagtcgccatcggccaggacctctagaccccagtcgctaaagctcagtgctggctacccagggaggggcctggactgaggtcctagaactctgctgaggccttgtagggactgagatggtgctacctggggcctggggcctggggcctggggcctggagcggggtgagccagggggaccgtagcagcctgtcaaagtggaagggtgttctgggcatctggaactgcatgcagtccaggctgagggccccagagaagtactgaggggctctgtgtccaaggccaagaagccacaggccaggcagaggagtggggcctggaccaggggtgggcactgaccaccagcacacgcagtcatcccgggcacaccttccttgtccaagccctcagggcaaaaggatcaaggaaattccccggagaaggagggcacctagcctgagtgatcatcctgtccccaccccggtccctacacagggacacaggcggggcccttctgggaataggctttcccagtgtctgccctgcgcagaaacagccccgaccctgaacctgcctgcccctccctttctaagacgcccgacatcctctgcacagagcatacggctcctaagtacaagacgactcgttcttgccgtggaaagttcgggaaaagacaaaaagatactgcaggaggaataaaatgccctccaaggtccccccacacccgggctcctcctccttctcctctcccggcggacggagtgctgatttggacacgtcactccctgtccctgaagggcctctccacgaccactgtgttgtgtcattgcggattctatttcttcctttgtctgcccttaattttttgaatgttcacaataaacatgcattactttcaaagtggaaaaaaatggatccactttatgaggaattcttttttatttaaaaacgtggcccaaggcagtggccgcccagtctggggtcggtccaagctggaagtcttttggtccaactggggcagggccagccactcaccctcccccaccgtgttcttcttcccgcctcctcctttaaaggtgccccagggccacgagccaccatctgtcacctgcctgccacctgccaccatgcgctgtcttgccagctctggcctctggcctctgatctcctctggaggctgctctttgcactggccccagccatctccttgccaggtaagcccaggaggggcatcctgccatccctctgctccaggtccccccaccccccgaaatgctgcccaggcctcacagtttggtga

SEQ ID NO: 13 - DNA, 1167 bp, Artificial Sequence
  whey acidic protein gene promoter
aggaaagcacactcgacactcgaacggactgcctactgtcagatcccatttacatgagatgcccagaatagacagacgcagaaaccgagcagagaggtagttgccaaggcctgggggctcggggaactagcgagaggctgctggcaggcacaggttttcctttggggctggcctgaaacgaaacatcaaggttacagcctgaaagagcttcccctgggactttgtcttcaaagaggagaggccatgggccacagtgaagacctccggccagtcaaaggagtatgggctgcaccataggctggcgcgacagccagtaaacacacagtcactcactctcgagtcattgcatccccttccttgcaagagaagtcaaggaaatgtcccgagagcaatgggcacagtgcccaacaggacatcccatccgggcccatgacaccgttggcacagcatggggcccttctgagaagtgggctttcaaggttccctgcacaggcaatccttttttgatgtgtaccctgtactctctacaaggagcaagtgcctccacattcttataaaactttttagaaaactccagaaaagcaccaagaaaagaaaccatcctctgatgtgactgtacacatttggagctcggaatttccttttttttttttttttaaagatttttatttatttcatgtatgggagcacactgtcgctatcttcagacacaccagaagagggcatcagatcccactggatcccagatggttgtgagccaccatgtggttgctgggacctgaactcaggacctctggaagagcagtcagtgctcccaaccactgagccatctctccagccctcggaatttcctttgtccgagaaaggggtcccaacccaaccattcaaagtgatatctgtcacatttgttacagatcccatttcttccttctctgctccttaatttttttcgttttggccataaacaagttttaccttttaagtgaaaaaataacgaccacccttacaaaggacttcttaaaaatggactccgaattgtgaaccttgttctggtagcctgggcctgctctctgcatgtgtccaagaggaagtgttttagcccatctacgcctatgcaagcctgcccccctccttccccaaagtcttcctcctgtgggtcctttaaat

SEQ ID NO: 14 - DNA, 195 bp, Artificial Sequence
  F-lactoglobulin gene promoter
ggcccagagggggacttcctgcttggccccggatggaagaaggcctcctattgtcctcgtagaggaagccaccccggggcccggggatgagccaagtaggattccgggaacctcgtggctggggcgcggcccgggctggctggctggcacgcctcctgtataaggccccgagcccgctgtctcagccctccactc

SEQ ID NO: 15 - DNA, 4204 bp, Artificial Sequence
  F-lactoglobulin gene promoter
gtcgacctgcaggtcaacggatctctgtgtctgttttcatgttagtaccacactgttttggtggctgtagctttcagctacagtctgaagtcataaagcctggtacctccagctctgttctctctcaagattgtgttctgctgtttgggtctttagtgtctccacacaatttttagaattgtttgttctagttctgtgaaaaatgatgctggtattttgataaggattgcattgaatctgtaaagctacagatatagtcattgggtagtacagtcactttaacaatattaactcttcacatctgtgagcatgatatattttccccctctatatcatcttcaattcctcctatcagtttctttcattgcagttttctgagtacaggtcttacacctccttggttagagtcattcctcagtattttattcctttgatacaattgtgaatgaggtaattttcttagtttctctttctgatagctcattgttagtgtatatatagaaaagcaacagatttctatgtattaattttgtatcctgcaacagatttctatgtattaattttgtatcctgctactttacggaattcacttattagctttttggtgacatcttgaggattttctgaagaaaatggcatggtatggtaggacaaggtgtcatgtcatctgcaaacagtggcagttttccttcttcccttccaacctggatttctttgatttctttctgtctgagtacgactaggattcccaatactataccgaataaaagtggcaagagtggacatccttgtcttatttttctgaccttagaggaaatgctttcagtttttcaccattaattataatgtttactgtgggcttgtcatatgtggccttcattatatggaggtctattccctctatacccaccttgttgagagtttttatcataaaagtatgttgaattttgtcaaaagtttttcctgcatctattgagatgatttttactcttcaattcattaatgatttttattcttcattttgttaatgatttccattcttcaatttgttaacgtggtatatcacattgattgatttgtggatacctttgtatccctgggataaacctcacttgatcatgagctttcaatgtatttttgaattcactttgctaatattctgttgggtatttttgcatctctattcatcaatgatattggcctaagaaaggttttgtctggttttagtatcagggtgatgctggcctcatagagagagtttagaagcatttcctcctctttgatttttcggaatagtttgagtaggataggtattaactcttctttaaatgtttggggacttccctggtgagccggtggttgagaatccgcctcagggatgtgggtttgatccctggtcagggaaccattaataagatcccacatgctgcaggcaacaagcccccaagctgcaaccactgagctgcaaccgctgcagtgcccacaggccacgaccagagaaagcccacatacagcagggaagacccagcacaaccggaaaaaggagtttggtggaatacagctgtgaagccgtctggtcctggactcctgcttgagggaattttttaaaaattattgattcaatttcattactggtaactggtctgttcatattttctatttcttccgggttcagtcttgggagattgtacatgcctaggaatgtgtccgtttcttctaggttgtccattttattggacatgcatgggagcacacagcaccgaccagcgagactcatgctggcttcctggggccaggctggggccccaagcagcatggcatcctagagtgtgtgaaagcccactgaccctgcccagccccacaatttcattctgagaagtgattccttgcttctgcacttacaggcccaggatctgacctgcttctgaggagcaggggttttggcaggacggggagatgctgagagccgacgggggtccaggtcccctcccaggcccccctgtctggggcagcccttgggaaagattgccccagtctccctcctacagtggtcagtcccagctgccccaggccagagctgctttatttccgtctctctctctggatggtattctctggaagctgaaggttcctgaagttatgaatagctttcgggtgaagggcatggtttgtggtcacggttcacaggaagcttgggagaccctgcagctcagacgtcccgagattggtggcacccagatttcctaagctcgctggggaacagggcgcttgtttctccctggctgacctccctcctccctgcatcacccagttctgaaagcagagcggtgctggggtcacagcctctcgcatctaacgccggtgtccaaaccacccgtgctggtgttcggggggctacctatggggaagggcttctcactgcagtggtgccccccgtcccctctgagatcagaagtcccagtccggacgtcaaacaggccgagctccctccagaggctccagggagggatccttgcccccccgctgctgcctccagctcctggtgccgcacccttgagcctgatcttgtagacgcctcagtctagtctctgcctccgtgttcacacgccttctccccatgtcccctccgtgtccccgttttctctcacaaggacaccggacattagattagcccctgttccagcctcacctgaacagctcacatctgtaaagacctagattccaaacaagattccaacctgaagttcccggtggatgtgagttctggggcgacatccttcaaccccatcacagcttgcagttcatcgcaaaacatggaacctggggtttatcgtaaaacccaggttcttcatgaaacactgagcttcgaggcttgttgcaagaattaaaggtgctaatacagatcagggcaaggactgaagctggctaagcctcctctttccatcacaggaaaggggggcctgggggcggctggaggtctgctcccgtgagtgagctctttcctgctacagtcaccaacagtctctctgggaaggaaaccagaggccagagagcaagccggagctagtttaggagacccctgaacctccacccaagatgctgaccagccagcgggccccctggaaagaccctacagttcaggggggaagaggggctgacccgccaggtccctgctatcaggagacatccccgctatcaggagattcccccaccttgctcccgttcccctatcccaatacgcccaccccacccctgtgatgagcagtttagtcacttagaatgtcaactgaaggcttttgcatcccctttgccagaggcacaaggcacccacagcctgctgggtaccgacgcccatgtggattcagccaggaggcctgtcctgcaccctccctgctcgggccccctctgtgctcagcaacacacccagcaccagcattcccgctgctcctgaggtctgcaggcagctcgctgtagcctgagcggtgtggagggaagtgtcctgggagatttaaaatgtgagaggcgggaggtgggaggttgggccctgtgggcctgcccatcccacgtgcctgcattagccccagtgctgctcagccgtgcccccgccgcaggggtcaggtcactttcccgtcctggggttattatgactcttgtcattgccattgccatttttgctaccctaactgggcagcaggtgcttgcagagccctcgataccgaccaggtcctccctcggagctcgacctgaaccccatgtcacccttgccccagcctgcagagggtgggtgactgcagagatcccttcacccaaggccacggtcacatggtttggaggagctggtgcccaaggcagaggccaccctccaggacacacctgtccccagtgctggctctgacctgtccttgtctaagaggctgaccccggaagtgttcctggcactggcagccagcctggacccagagtccagacacccacctgtgcccccgcttctggggtctaccaggaaccgtctaggcccagaggggacttcctgcttggccttggatggaagaaggcctcctattgtcctcgtagaggaagccaccccggggcctgaggatgagccaagtgggattccgggaaccgcgtggctgggggcccagcccgggctggctggcctgcatgcctcctgtataaggccccaagcctgctgtctcagccctcc

SEQ ID NO: 16 - DNA, 3834 bp, Artificial Sequence
  F-lactoglobulin gene promoter
gtcaacggatctctgtgtctgttttcatgttagtaccatactgttttggtggctgtagctttgagctatagtctgaagtcataaagcccgatacctccagctctgttcttctttctcaagattgtgttctgctgtttgggtctttagtgtctccacacaatttttagaattgtgtgttctagttctgtgaaaaatgatgctggcattttgataaggattgcattgaatctgtaaagctacagatatagtcattgggtagtacaatcactttaacaatattaactcttcaaatccgtgagcatgatgtattttccccctccatatcatcttcaattccttctatcagtttctttcattgcagttttctgagtataggtcttacacctccttgattagagtcattcctcagtattttattcctttgatacaattgtgaatgagatcattttcttagtttctctttctgatagcccattgttagtgtatagaaaagcaacagagttctatgtattaattttgtatcctgcaacagatttctatgtattaattttgtatcctgctactttactgaatttacttattagctttttggtgacatcttaaggattttcttaagaaaatggcatggtatggtaggacaaggtgtcacgtcatctgcaaacagtggcagttttacttcttcccttccagcctggatttctttgatttctttctgtctgagtactgtgactaggattcccaatactataccgaacaaaagtggcaagagtggacatccttgtcttatttttctgaccttagaggaaatgctttcagtttttcaccattaattataatgtttactgtgggcttgtcatatgtggccttcattatatggaggtctattccctctatacccactttgttgagagtttttatcatgaaagtatgttgaattttgtcaacagtttttcctgcatctattgagatgatttttactcttcaattcattaatgatttttattcttcattttgttaatgatttccattcttcaatgtgttaacgtggtatatcacattgattgatttgtggatatctttgtatccctgggataaacctcacctgatcatgagctttcaatgtatttttgaattcactttgctaatattctgctgggtatttttacatctctattcatcaatgatattgacctaagattttctttctttttttttttgtaaagtttttgtgtggttttagtatcagggtgatgctggcctcatagagagagtttagaagcatttcctcctctttgattttttggaatagtttgagtaggataggtattaactcttctttaaatgtttggggacttccctggtgagccggtggttgagaatccgcctcagggatgtgggtttgatccctggtcagggaaccattaataagctcccacatgctgcagggcaacaagcccccaagctgcaaccactgagctgcaaccgctgcagtgcccacgggccacgaccagagaaagcccacatacagcagggaagacccagcacaacctaaaaaaggagtttggtggaatacagctgtgaagccatctggtcctggactcctgcttgagggaatttttttaaaattattgattcaatttcattactgattgccccagtctccctcccacagtggtcagtcccagctgccccaggccagaggtgctttatttccgtctctctctctggatggtattctttggaagctgaagattcctggaagttatgaatagcttcgccctgaagggcatggtttatggtcacggttcacaggaacttgggagaccctgcagctcagacgtcccgaggttggtggcacccagatttcctaagctcgctggggaagggggcgcttgtttctccctggctgacctccctccgccctgcatcacccagttctgagagcagagcggtgctggggggcacagcctctcgcatctgacgccggtgtccaaaccacccgtgctggtgttcggggggctacctatggggaagggctcctcactgcaggggtgccccccgtcccctctgagatcagaagtcccagtccggacagcgaacaggccaagctccctccagaggctccaggaagggatccttgccccccgccgccgcctccagctcctggtgccgcacccttgagcctgatcttgtagacgcctcagtctagtctctgcctccgtgttcacatgccttctccccatgtcccctccatgtccccgttttctctcacaaggacaccggacagtagattagcccctgttccagcctcacctgaacagctcacatctgtaaagacctagattccaaacaagattccaacctgaagttcctggtggatgtgagttctggggcaacatccttcaaccccatcacagcttgcagttcatcacaaaacatggaacctggggtttatcataaaacctaggttcttcatgaaacactgagcttcgaggcttgttgcaagaattaaaggtgctaatacagatcaaggcaaggactgaagctggccaagcctactctttccatcacaggaaaggggggtctgggggcggctgggggtctgctcccgtgagtgagctcttttctgctacagtcaccaacagtctctccgggaaggaaaccagaggccagagagcaagccagagctagtttaggagacccccgaacctccaaccaagatgctgaccaggccagcgggccccctggaaagaccctacagttcaggggggaagaggggctgacccgccaggtccctgctatcaggagacatccccgctatcaggagattcccccaccttgctcccgttccgctaccccaatacgcccaccccacccctgtgatgagtggtttagccacttagaatgtcaactgaaggcttttgcaccctctttgccagaggcacaaggcacccacagcccgctgggtaccaacgcccatgtggattcagccaggaggcctgtcctgcaccctccctgctcgggccccttctgtactcagcaacacacccagcaccagcattcccactgctcctgaggtctgcaggcagctcgctgtagcctgagcggtgtggagggaagtgtcctgggagacttaaaatgtgggaggtgggagggggggaggttgggccctgtgggcctgcccaccccgtgtgcctgcatggagccccagtgctgctcagccgtgcccccgccgcaggggtcaggtcactttcccgtcctgggggttattatgaccgttgtcattttcattgccatttttgctaccctaactgggcagcaggtgcttgcagagccctcgataccgaccaggtcccccctcggagctccacctgaaccccgtgtcacccttgccccagcctgcagaggatggggtcactgcagagatcccttcacccaaggccacggtcacatggtttggaggagctggtgcccaaggcagaggccaccctctaggacacacctgtccccagtgctggctctgacctgcccttgtctaagaggctgaccccggaagtgttcctggcactggcagccagcctgacccagagtccagacacccacctgtgcccccacttctggggtctaccaggaaccgtctaggcccagagggggacttcctgcttggccccggatggaagaaggcctcctattgtcctcgtagaggaagccaccccggggcccggggatgagccaagtaggattccgggaacctcgtggctgggggcccggcccgggctggctggctggcacgcctcctgtataaggccccgagcccgctgtctcagccctccg

SEQ ID NO: 17 - DNA, 20 bp, Artificial Sequence
  FABPi promoter region F primer
gaattccttaatttgcataa

SEQ ID NO: 18 - DNA, 24 bp, Artificial Sequence
  FABPi promoter region R primer
ctcgagcagctgtgtcatagttct